SEQUENCE LISTING (29 sequences)

SEQ ID NO: 1 - protein, 8 aa, Artificial Sequence
  Amino acid sequence of murine anti hFcgRI.1 CDR1 VH
GFSLTTYG

SEQ ID NO: 2 - protein, 7 aa, Artificial Sequence
  Amino acid sequence of murine anti hFcgRI.1 CDR2 VH
IWSGGST

SEQ ID NO: 3 - protein, 7 aa, Artificial Sequence
  Amino acid sequence of murine anti hFcgRI.1 CDR3 VH
AREWFAY

SEQ ID NO: 4 - protein, 6 aa, Artificial Sequence
  Amino acid sequence of murine anti hFcgRI.1 CDR1 VL
ENIYSY

SEQ ID NO: 5 - protein, 3 aa, Artificial Sequence
  Amino acid sequence of murine anti hFcgRI.1 CDR2 VL
SAK

SEQ ID NO: 6 - protein, 9 aa, Artificial Sequence
  Amino acid sequence of murine anti hFcgRI.1 CDR3 VL
QHHYGTPYT

SEQ ID NO: 7 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 8 - protein, 314 aa, Homo sapiens
  NATURAL HUMAN RECEPTOR FcgRIIA = AAH20823.1 (variant H131) (1)..(314)
METQMSQNVCPRNLWLLQPLTVLLLLASADSQAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGVIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 9 - protein, 113 aa, Artificial Sequence
  Amino acid sequence of murine heavy chain variable region (VH) of anti-hFcgRI.1
EVQLQESGPGLVQPSQSLSITCTVSGFSLTTYGVHWVRQSPGKGLEWLGMIWSGGSTDYNAAFISRLSISKDNSKSQVFFKMNSLQADDTAIYYCAREWFAYWGQGTSVTVSS

SEQ ID NO: 10 - protein, 107 aa, Artificial Sequence
  Amino acid sequence of murine light chain variable region (VL) of anti- hFcgRI.1
ELVMTQSPASLSASVGETVTITCRASENIYSYLAWYQQKQGKSPQLLVYSAKTLAEGVPSRFSGSGSGTQFSLKINSLQPEDFGSYYCQHHYGTPYTFGGGTKMEIK

SEQ ID NO: 11 - protein, 8 aa, Artificial Sequence
  Amino acid sequence of murine anti hFcgRIIA CDR1 VH
GYTFTNYG

SEQ ID NO: 12 - protein, 8 aa, Artificial Sequence
  Amino acid sequence of murine anti hFcgRIIA CDR2 VH
LNTYTGES

SEQ ID NO: 13 - protein, 13 aa, Artificial Sequence
  Amino acid sequence of murine anti hFcgRIIA CDR3 VH
ARGDYGYDDPLDY

SEQ ID NO: 14 - protein, 11 aa, Artificial Sequence
  Amino acid sequence of murine anti hFcgRIIA CDR1 VL
KSLLHTNGNTY

SEQ ID NO: 15 - protein, 4 aa, Artificial Sequence
  Amino acid sequence of murine anti hFcgRIIA CDR2 VL
RMSV

SEQ ID NO: 16 - protein, 9 aa, Artificial Sequence
  Amino acid sequence of murine anti hFcgRIIA CDR3 VL
MQHLEYPLT

SEQ ID NO: 17 - protein, 120 aa, Artificial Sequence
  Amino acid sequence of heavy chain variable region (VH) of anti- hFcgRIIA
EIQLQQSGPELKKPGETVKISCKASGYTFTNYGMNWVKQAPGKGLKWMGWLNTYTGESIYPDDFKGRFAFSSETSASTAYLQINNLKNEDMATYFCARGDYGYDDPLDYWGQGTSVTVSS

SEQ ID NO: 18 - protein, 112 aa, Artificial Sequence
  Amino acid sequence of light chain variable region (VL) of anti- hFcgRIIA
DVVMTQTPPSVPVTPGESVSISCRSSKSLLHTNGNTYLHWFLQRPGQSPQLLIYRMSVLASGVPDRFSGSGSGTAFTLSISRVEAEDVGVFYCMQHLEYPLTFGAGTKLELK

SEQ ID NO: 19 - protein, 460 aa, Artificial Sequence
  Amino acid sequence of chimeric anti-FcgRI heavy chain (V mouse, C human)
MGDNDIHFAFLSTGAHSEVQLQESGPGLVQPSQSLSITCTVSGFSLTTYGVHWVRQSPGKGLEWLGMIWSGGSTDYNAAFISRLSISKDNSKSQVFFKMNSLQADDTAIYYCAREWFAYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSREEMTKNQVSLTCLVKGFYPSDIAVEWVSNGQPENNYKTTPPVLDSDGSFFLYSKLTVDKSRWQQGNVFSCSVLHEALHNHYTQKSLSLSPGK

SEQ ID NO: 20 - protein, 231 aa, Artificial Sequence
  Amino acid sequence of chimeric anti-FcgRI light chain (V mouse, C human)
MGDNDIHFAFLSTGAHSELVMTQSPASLSASVGETVTITCRASENIYSYLAWYQQKQGKSPQLLVYSAKTLAEGVPSRFSGSGSGTQFSLKINSLQPEDFGSYYCQHHYGTPYTFGGGTKMEIKRTAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 467 aa, Artificial Sequence
  Amino acid sequence of chimeric anti-FcgRIIA heavy chain sequence (V mouse, C human)
MGDNDIHFAFLSTGAHSEIQLQQSGPELKKPGETVKISCKASGYTFTNYGMNWVKQAPGKGLKWMGWLNTYTGESIYPDDFKGRFAFSSETSASTAYLQINNLKNEDMATYFCARGDYGYDDPLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSREEMTKNQVSLTCLVKGFYPSDIAVEWVSNGQPENNYKTTPPVLDSDGSFFLYSKLTVDKSRWQQGNVFSCSVLHEALHNHYTQKSLSLSPGK

SEQ ID NO: 22 - protein, 236 aa, artificial sequence
  Amino acid sequence of chimeric anti-FcgRIIA light chain (V mouse, C human)
MGDNDIHFAFLSTGAHSDVVMTQTPPSVPVTPGESVSISCRSSKSLLHTNGNTYLHWFLQRPGQSPQLLIYRMSVLASGVPDRFSGSGSGTAFTLSISRVEAEDVGVFYCMQHLEYPLTFGAGTKLELKRTAAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 1383 bp, Artificial Sequence
  DNA sequence encoding chimeric anti-FcgRI heavy chain (V mouse, C human)
atgggtgacaatgacatccactttgcctttctctccacaggcgcgcactccgaggtgcagcttcaggagtcaggacctggcctagtgcagccctcacagagcctgtccatcacctgcacagtctctggtttctcattgactacctatggtgtacactgggttcgccagtctccaggaaagggtctggagtggctgggaatgatatggagtggtggaagcacagactataatgcagctttcatatccagactgagcatcagcaaggacaactccaagagccaagttttctttaaaatgaacagtctgcaagctgatgacacagccatatactactgtgccagagaatggtttgcttactggggccaaggaacctcagtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggtgagcaatgggcagccggagaacaactacaagaccacgcctcccgtgctggactccgacggctccttcttcctctatagcaagctcaccgtggacaagagcaggtggcagcaggggaacgtcttctcatgctccgtgttacatgaggctctgcacaaccactacacgcagaagagcctctccctgtccccgggtaaatga

SEQ ID NO: 24 - DNA, 696 bp, Artificial Sequence
  DNA sequence encoding chimeric anti-FcgRI light chain (V mouse, C human)
atgggtgacaatgacatccactttgcctttctctccacaggcgcgcactccgagctcgtgatgacacagtctccagcctccctatctgcatctgtgggagaaactgtcaccatcacatgtcgagcaagtgagaatatttacagttatttagcatggtatcagcagaaacagggaaaatctcctcagctcctggtctatagtgcaaaaaccttagcagaaggtgtgccatcaaggttcagtggcagtggatcaggcacacagttttctctgaagatcaacagcctgcagcctgaagattttgggagttattactgtcaacatcattatggtactccgtacacgttcggaggggggaccaagatggaaataaaacgcacggcggccgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgctaa

SEQ ID NO: 25 - DNA, 1404 bp, Artificial Sequence
  DNA sequence encoding chimeric anti-FcgRIIA heavy chain seqence (V mouse, C human)
atgggtgacaatgacatccactttgcctttctctccacaggcgcgcactccgagatccagctgcagcagtctggacctgagctgaagaagcctggagagacagtcaagatctcctgcaaggcttctgggtataccttcacaaactatggaatgaactgggtgaagcaggctccaggaaagggtttaaagtggatgggctggttaaacacctacactggagagtcaatatatcctgatgacttcaagggacggtttgccttctcttcggaaacctctgccagcactgcctatttgcaaatcaacaacctcaaaaatgaggacatggctacatatttctgtgcaagaggggactatggttacgacgaccctttggactactggggtcaaggaacctcagtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtcgacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggtgagcaatgggcagccggagaacaactacaagaccacgcctcccgtgctggactccgacggctccttcttcctctatagcaagctcaccgtggacaagagcaggtggcagcaggggaacgtcttctcatgctccgtgttacatgaggctctgcacaaccactacacgcagaagagcctctccctgtccccgggtaaatga

SEQ ID NO: 26 - DNA, 711 bp, Artificial Sequence
  DNA sequence encoding chimeric anti-FcgRIIA light chain (V mouse, C human)
ggtgacaatgacatccactttgcctttctctccacaggcgcgcactccgatgttgtgatgacccagactccaccctctgtacctgtcactcctggagagtcagtatccatctcctgcaggtctagtaagagtctcctgcatactaatggcaacacttacttgcattggttcctacagaggccaggccagtctcctcagctcctgatatatcggatgtccgtccttgcctcaggagtcccagacaggttcagtggcagtgggtcaggaactgctttcacactgagcatcagtagagtggaggctgaggatgtgggtgttttttactgtatgcaacatctagaatatccgctcacgttcggtgctgggaccaagctggaactgaaacgcacggcggccgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgctaataa

SEQ ID NO: 27 - protein, 314 aa, Homo sapiens
  amino acid sequence of human FcgRIIA variant R131 (1)..(314)
METQMSQNVCPRNLWLLQPLTVLLLLASADSQAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSRLDPTFSIPQANHSHSGDYHCTGNIGYTLFSSKPVTITVQVPSMGSSSPMGVIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 28 - DNA, 339 bp, Artificial Sequence
  DNA sequence encoding VH (Heavy chain variable region) of anti-FcgRI.1
gaggtgcagcttcaggagtcaggacctggcctagtgcagccctcacagagcctgtccatcacctgcacagtctctggtttctcattgactacctatggtgtacactgggttcgccagtctccaggaaagggtctggagtggctgggaatgatatggagtggtggaagcacagactataatgcagctttcatatccagactgagcatcagcaaggacaactccaagagccaagttttctttaaaatgaacagtctgcaagctgatgacacagccatatactactgtgccagagaatggtttgcttactggggccaaggaacctcagtcaccgtctcctca

SEQ ID NO: 29 - DNA, 321 bp, Artificial Sequence
  DNA sequence encoding VL of anti-FcgRI.1
gagctcgtgatgacacagtctccagcctccctatctgcatctgtgggagaaactgtcaccatcacatgtcgagcaagtgagaatatttacagttatttagcatggtatcagcagaaacagggaaaatctcctcagctcctggtctatagtgcaaaaaccttagcagaaggtgtgccatcaaggttcagtggcagtggatcaggcacacagttttctctgaagatcaacagcctgcagcctgaagattttgggagttattactgtcaacatcattatggtactccgtacacgttcggaggggggaccaagatggaaataaaa

(19) United States
(12) Patent Application Publication
(10) Pub. No.: US 2015/0322152 A1
(43) Pub. Date: Nov. 12, 2015

(54) USE OF ANTI-FCYRI AND/OR ANTI-FCYRIIA ANTIBODIES FOR TREATING ARTHRITIS, INFLAMMATION, THROMBOCYTOPENIA AND ALLERGIC SHOCK

(71) Applicant: INSTITUT PASTEUR, Paris (FR)

(72) Inventors: Pierre BRUHNS, Paris (FR); David MANCARDI, Paris (FR); Friederike JONSSON, Antony (FR); Pierre LAFAYE, Malakoff (FR)

(21) Appl. No.: 14/648,293

(22) PCT Filed: Nov. 30, 2012
(86) PCT No.: PCT/IB2012/003135

§ 371 Date: May 29, 2015

(51) Int. Cl. C07K 16/28

(52) U.S. Cl. CPC C07K 16/283; C07K 2317/565; C07K 2317/76

(57) ABSTRACT

The present invention relates to the prevention and/or treatment of arthritic, inflammatory and/or allergic reactions as well as thrombocytopenia, by blocking the human receptors FcyRI and FcyRIIA. It is disclosed a mouse monoclonal antibody (mAb) which efficiently blocks human FcyRI and can therefore be used, alone or in a combination product, for example with an anti-hFcyRIIA blocking antibody, in the prevention and/or the treatment of these diseases.

### BACKGROUND OF THE INVENTION

Inflammation plays a fundamental role in host defenses and the progression of immune-mediated diseases. The inflammatory response is initiated in response to injury (e.g., trauma, ischemia, and foreign particles) and/or infection (e.g., bacterial or viral infection) by a complex cascade of events, including chemical mediators (e.g., cytokines and prostaglandins) and inflammatory cells (e.g., leukocytes). When the inflammatory response is uncontrolled, or is due to autoimmune disorders, inflammatory diseases may arise. These inflammatory diseases are, for example, arthritis, related arthritic conditions (e.g., osteoarthritis, rheumatoid arthritis, and psoriatic arthritis), inflammatory bowel disease (e.g., Crohn's disease and ulcerative colitis), psoriasis, atopic dermatitis, contact dermatitis, chronic obstructive pulmonary disease, and chronic inflammatory pulmonary diseases.

In particular, Rheumatoid arthritis (RA) is a chronic, systemic inflammatory disorder that may affect many tissues and organs, but principally attacks flexible (synovial) joints. The pathology of the disease process often leads to the destruction of articular cartilage and ankylosis (fusion) of the joints. About 1% of the world's population is afflicted by rheumatoid arthritis, women three times more often than men. Onset is most frequent between the ages of 40 and 50, but people of any age can be affected. It can be a disabling and painful condition, which can lead to substantial loss of functioning and mobility if not adequately treated. There is no known cure for rheumatoid arthritis, although many different types of treatment induce pain relief. Existing “treatments” are mostly not inducing a healing effect, but rather only mask symptoms or at the best slow down the progress of the disease.

Allergic inflammation is an important pathophysiological feature of several medical conditions including allergic asthma, atopic dermatitis, allergic rhinitis and several ocular allergic diseases. The most severe manifestation of allergic complications, the anaphylactic shock, can be mortal, even in young patients. Anaphylaxis is estimated to be responsible for more than 1,500 deaths per year in the United States. Despite all the effort of the researchers, the current treatments of anaphylaxis protect from death in 50% of the cases only.

In human, thrombocytopenia (or thrombopenia) is a relative decrease of platelets in blood. One common definition of thrombocytopenia is a platelet count below 50,000 per microlitre.

There is a significant need for safe and effective methods of treating, preventing and managing inflammation-related disease such as arthritis and allergies, particularly for patients that are refractory to conventional treatments. In addition, there is a need to treat these diseases while reducing or avoiding the toxicity and/or side effects associated with conventional therapies.

In this context, the present inventors investigated the role of two human IgG receptors, hFcγRI and hFcγRIIA, in antibody-mediated models of these diseases. Interestingly, they found that hFcγRIIA triggers airway inflammation and systemic anaphylaxis (Jonsson F. et al, Blood 2012; 119:2533-2544). They also found that hFcγRI induces airway inflammation, systemic anaphylaxis, autoimmune arthritis and thrombocytopenia (results below). More importantly, the present inventors have shown that it is possible to prevent and even abolish these diseases by blocking any of these human receptors or both, for example with blocking antibodies.

The inventors identified an anti-FcγRI antibody inducing very good therapeutic response. This antibody is hereafter caller “anti-hFcγRI.1” or “the antibody of the invention” or “RI.1”.

This antibody binds specifically (Kₐ=1.5×10⁹M⁻¹) the human FcγRI receptor of SEQ ID NO:7 (NP_{—}000557) (FIG. 7), which is the product of the FcγRIA gene located on human chromosome 1q21.1 and corresponds to the 374 amino acids of the high-affinity immunoglobulin gamma Fc receptor I precursor [Homo sapiens]. Note that this antibody or fragment may also bind to FcγRI from other non-human species (e.g., other mammals and vertebrates). However, this antibody does not bind to any of the other human IgG receptors such as FcγRIIA, FcγRIIB, FcγRIIC, FcγRIIIA or FcγRIIIB.

Receptors for the Fc portion of IgG (FcγR) are expressed at the surface of certain human and murine cells which contribute to the protective functions of the immune system. They bind the constant regions (Fc) of the IgG antibodies.

Human FcγRI is the only high-affinity IgG receptor in humans. hFcγRI binds human IgG1, IgG3 and IgG4 with a high affinity and has no affinity for IgG2 (Bruhns P. et al, Blood 2009; 113:3716-3725). Structurally, hFcγRI is composed of a signal peptide that allows its transport to the surface of a cell, a hydrophobic transmembrane domain, a short cytoplasmic tail and three extracellular immunoglobulin domains of the C2-type that are used to bind antibody. As mentioned previously, human FcγRI is expressed at the surface of several cell types in human: blood monocytes, dendritic cells, neutrophils and tissue macrophages.

Fc gamma receptors generate signals within the cells that carry them through an Immunoreceptor tyrosine-based activation motif (ITAM), an important activation motif having a specific sequence of amino acids (Yxx(L/I)) occurring twice in close succession in the intracellular tail of a receptor. When the tyrosine (Y) residue of the ITAM is phosphorylated, a signaling cascade is generated within the cell. This phosphorylation reaction typically follows interaction of Fc receptors with multimeric ligand, thus inducing Fc receptor aggregation.

hFcγRI does not have an ITAM in its intracellular part but can transmit an activating signal by interacting with another protein that does. This adaptor protein is called the Fcγ subunit (FcRγ) which contains the two ITAMs. Once aggregated, hFcγRI can induce phagocytosis, cell activation, cell degranulation, cytokine release, microbe killing and the activation of the respiratory burst.

The human receptor hFcγRI is also known as the human cluster of differentiation 64 (CD64). As used herein, the terms “human CD64”, “human high-affinity IgG receptor” and “human Fc gamma receptor I” (hFcγRI) are used interchangeably.

hFcγRI (CD64), hFcγRIIB (CD32B) and hFcγRIIIA(CD16A) exist in both human and murine species. hFcγRIIA (CD32A), hFcγRIIC (CD32C) and hFcγRIIIB (CD16B) are specific to humans, whereas FcγRIV is specific to mice. This nomenclature is based on amino acid sequence homology but does not systematically reflect functional homologies or similar expression pattern between FcγRs in both species (Bruhns P. Blood 2012, 119(24):5640-5649). It is now widely accepted that the role of human FcγRs cannot be predicted from the role of their homologs studied in mice (Smith P, et al., Proc Natl Acad Sci USA. 2012, 109(16):6181-6; Nimmerjahn F, et al. Nat Rev Immunol. 2008, 8(1):34-47; Bruhns P. et al Blood. 2012, 119(24):5640-5649).

In particular, it has been demonstrated that the mouse high-affinity IgG receptor mFcγRI has an expression pattern restricted to monocyte-derived dendritic cells (Langlet C, et al. J Immunol. 2012, 188(4):1751-1760; Mancardi D A, et al. J Immunol. 2011, 186(4):1899-1903; Tan P S, et al. J Immunol. 2003, 170(5):2549-2556). On the contrary, the expression pattern of hFcγRI is not restricted to dendritic cells and extends to blood monocytes and tissue macrophages, therefore differing from its mouse homolog mFcγRI. Moreover, the human receptor hFcγRI is also expressed by neutrophils in most inflammatory contexts (Quayle J A, et al. Immunology 1997, 91(2):266-273 and Cid J, et al. J Infect. 2010, 60(5):313-319). Thus, the expression pattern of human and mouse FcγRI appear very different and suggest that their roles in pathology and therapy may also be very different. Surprisingly, the role(s) of hFcγRI on monocytes, macrophages and neutrophils, has not been addressed so far.

### SUMMARY OF THE INVENTION

In this context, the present inventors studied the role of hFcγRI in antibody-mediated models of disease in vivo, in particular on hFcγRI-transgenic mice that are deficient for multiple endogenous FcRs. They demonstrated that hFcγRI is involved in airway inflammation, systemic anaphylaxis, autoimmune arthritis and thrombocytopenia. More importantly, they show that it is possible to abolish these symptoms by efficiently blocking the hFcγRI receptor, for example with the monoclonal antibody of the invention (anti-hFcγR1.1).

Surprisingly, the novel monoclonal antibody (RI.1) isolated by the inventors is a blocking antibody which is capable of preventing interaction of the human FcγRI receptor with its natural ligand(s). More precisely, the present inventors have shown that this antibody anti-hFcγR1.1 efficiently blocks the binding of IgGs on the human FcγRI receptor (e.g., IgG2, see FIG. 7) and is therefore an antagonist of this receptor. RI.1 has been shown to successfully abolish airway inflammation, systemic anaphylaxis, autoimmune arthritis and thrombocytopenia in transgenic mice models (see the results below).

The inventors identified the Complementary Determining Regions (CDRs) of RI.1 as being, for the heavy chain and the light chain respectively, GFSLTTYG (V_{H}-CDR1), IWSGGST (V_{H}-CDR2), AREWFAY (V_{H}-CDR3), ENIYSY (V_{L}-CDR1), SAK (V_{L}-CDR2), QHHYGTPYT (V_{L}-CDR3) (SEQ ID NO:1 to 6 respectively). Moreover, the heavy chain variable region (V_{H}) of anti-hFcγRI.1 has the amino acid sequence SEQ ID NO: 9 and the light chain variable region (V_{L}) of anti-hFcγRI.1 has the amino acid sequence SEQ ID NO: 10.

Furthermore, it has been observed that the blocking of two IgG receptors (demonstrated for hFcγRI and FcγRIV, see FIGS. 2A, 3A, 4D, 5B, 6B) has an additive effect to reduce the symptoms of these diseases. The same principle should apply to hFcγRI and hFcγRIIA for example with the anti-hFcγRI.1 mAb and the known monoclonal antibody named IV.3 (which is further detailed below). The use of these two antibodies—alone or in a combination product-represents a novel therapeutic solution in the prevention and/or treatment of arthritic, inflammatory and allergic reactions and thrombocytopenia.

In a first aspect, the present invention relates to an antibody or a functional fragment thereof, which binds and blocks the human FcγRI receptor, said antibody comprising six Complementary Determining Regions (CDRs) consisting of SEQ ID NO:1-6. Preferably, said antibody comprises:
a) a heavy chain comprising three CDRs having the following amino acid sequences:

i) the heavy chain CDR1:
(SEQ ID NO: 1)
GFSLTTYG;
ii) the heavy chain CDR2:
(SEQ ID NO: 2)
IWSGGST;
iii) the heavy chain CDR3:
(SEQ ID NO: 3)
AREWFAY,

and
b) a light chain comprising three CDRs having the following amino acid sequences:

i) the light chain CDR1:
(SEQ ID NO: 4)
ENIYSY;
ii) the light chain CDR2:
(SEQ ID NO: 5)
SAK;
iii) the light chain CDR3:
(SEQ ID NO: 6)
QHHYGTPYT.

More preferably, said antibody comprises a heavy chain variable region (V_{H}) having the amino acid sequence SEQ ID NO: 9 and/or a light chain variable region (V_{L}) having the amino acid sequence SEQ ID NO: 10.

The present invention also relates to a humanized antibody or a functional fragment thereof, comprising six Complementary Determining Regions (CDRs) consisting of SEQ ID NO:1-6, and preferably the heavy chain variable region and light chain variable region of SEQ ID NO:19 and SEQ ID NO:20 respectively.

In a second aspect, the present invention pertains to this antibody (or a humanized form or functional a fragment thereof) for use for preventing and/or treating antibody-dependent inflammatory and autoimmune disorders, such as arthritic symptoms, allergic reactions, lupus or antibody-nephritis or for use for preventing and/or treating thrombocytopenia.

In a third aspect, the present invention pertains to a therapeutic substance combination product containing the antibody of the invention, or a functional fragment thereof, and a compound blocking the human FcγRIIA receptor. Preferably, said compound is a monoclonal antibody comprising:
a) a heavy chain comprising three CDRs having the following amino acid sequences:

(SEQ ID NO: 11)
i) the heavy chain CDR1: GYTFTNYG;
(SEQ ID NO: 12)
ii) the heavy chain CDR2: LNTYTGES;
(SEQ ID NO: 13)
iii) the heavy chain CDR3: ARGDYGYDDPLDY,

and
b) a light chain comprising three CDRs having the following amino acid sequences:

(SEQ ID NO: 14)
i) the light chain CDR1: KSLLHTNGNTY;
(SEQ ID NO: 15)
ii) the light chain CDR2: RMSV;
(SEQ ID NO: 16)
iii) the light chain CDR3: MQHLEYPLT.

More preferably, said compound is a monoclonal antibody comprising a heavy chain variable region (V_{H}) having the amino acid sequence SEQ ID NO: 17 and/or a light chain variable region (V_{L}) having the amino acid sequence SEQ ID NO: 18.

Even more preferably, said compound is a humanized antibody comprising six Complementary Determining Regions (CDRs) consisting of SEQ ID NO:11-16, and preferably the heavy chain variable region and light chain variable region of SEQ ID NO:21 and SEQ ID NO:22 respectively.

In a fourth aspect, the present invention pertains to the therapeutic substance combination product of the invention, for use for preventing and/or treating antibody-dependent inflammatory and autoimmune disorders, such as arthritic symptoms, allergic reactions, lupus or antibody-nephritis or for use for preventing and/or treating thrombocytopenia.

### FIGURE LEGENDS

FIG. 1. hFcγRI conserves its properties as a high-affinity IgG receptor in transgenic mice. (A-B) Representative histogram plots of hFcγRI expression on indicated cell populations from (A) blood or tissues of hFcγR^{tg} 5KO mice or (B) blood of normal human donors (two representative histogram plots from two different donors (#1 and #2) are represented for hFcγRI expression on neutrophils). (C) Histograms show the expression of the respective FcγRs (FLAG), or the binding of indicated mouse monomeric IgG to FLAG-tagged FcγR+CHO transfectants. Solid gray histograms represent the binding of secondary Abs alone. (D) Histograms show the expression of the respective FcγRs (FLAG), or the binding of indicated IgG ICs (black line) or Ag alone (solid gray histograms) to FcγR⁺ CHO transfectants, as revealed by neutravidin staining. (E) Real-time SPR sensorgrams and affinity constants were determined from SPR analysis. (E) Data correspond to the injection of 125 nM of hIgG1 (black) or of mIgG2a (grey) onto immobilized hFcγRI.

FIG. 2. hFcγRI can trigger inflammatory Arthritis in transgenic mice. (A-C) K/B×N PA in indicated mice injected with indicated mAbs (A,B, n=3; C, n=4). (D) Arthritis induced in anti-FcγRIV-treated hFcγRI^{tg} 5KO mice by K/B×N serum (n=4) or 80 μg of purified K/B×N IgG1 (n=3) or of purified K/B×N IgG2 (n=4). (E) K/B×N PA in anti-FcγRIV-treated hFcγR^{tg} 5KO mice injected with indicated liposomes (n=3) or mAbs (n=4).

FIG. 3. hFcγRI can trigger IC-induced airway inflammation in transgenic mice. (A,B) Neutrophil (A) count and (B) percentage among leukocytes. (C,D) Alveolar macrophage (C) count and (D) percentage among leukocytes. (E) MPO level and (F) hemorrhage score in BAL from hFcγRI^{tg} 5KO mice following injection of indicated reagents. IC stands for OVA injected i.v. followed by anti-OVA antiserum injected i.n. (n=4 in all groups).

FIG. 4. In vivo aggregation of hFcγRI induces passive systemic anaphylaxis. (A-B) Indicated mice were injected with (A) 200 μg of anti-hFcγRI.1 blocking mAb or anti-hFcγRI.2 non-blocking mAb, or (B) with indicated amount of anti-hFcγRI.2 non-blocking mAb and central temperatures were monitored (n≧3). The same curve corresponding to 200 μg anti-hFcγRI.2 non-blocking mAb injected in hFcγRI^{tg} 5 KO mice is represented in experiments A and B that were performed together. Note: anti-hFcγRI.1 mAb is an antagonistic blocking antibody and anti-hFcγRI.2 mAb an agonistic non-blocking antibody.

(C-D) 5KO and/or hFcγRI^{tg} 5 KO mice were pretreated with indicated reagents and injected with preformed mouse IC made of polyclonal anti-GPI serum and GPI, and central temperatures were monitored (C, n≧4; D, n≧3).

FIG. 5. Neutrophils are necessary for hFcγRI-dependent active systemic anaphylaxis. Indicated mice were immunized with BSA in Freund's adjuvant, challenged with BSA and central temperatures and survival rates were monitored. (A-B) ASA in hFcγR^{tg} 5KO or 5KO mice injected with indicated reagents (n=5). (C-F) ASA in anti-FcγRIV-treated hFcγRI^{tg} 5KO mice injected with indicated reagents (C, D, n=5; E, n=5; F, n≧3). Abbreviations: toxic liposomes (Cld2 lipo.); gadolinium chloride (GdCl3); cyproheptadine (cypro.)

FIG. 6. Macrophages are necessary for hFcγRI-dependent thrombocytopenia. (A) hFcγRI^{tg} 5KO (black) or 5KO (gray) mice were pretreated with indicated reagents before being injected i.v. with anti-platelet mAb (α-PLA). Platelet counts were acquired in blood at (left) indicated times presented as curves or (right) at t=4 hours presented as histograms, following α-PLA injection (n=3). (B) hFcγR^{tg} 5KO mice were pretreated with indicated reagents and platelet counts acquired in blood at t=4 hours following α-PLA injection (n=3). (C-E) 5KO mice (small histograms in inserts) or anti-FcγRIV-treated hFcγR^{tg} 5KO mice (large histograms, left in each panel) were pretreated with indicated reagents or splenectomized when indicated, and platelet counts acquired in blood at t=4 hours following α-PLA injection (C, D: n=3; E:

FIG. 7. Blocking of the hFcγRI receptor by the antibody of the invention. Histograms show the binding of (left column) anti-FLAG mAb or (all other columns) FITC-conjugated mIgG2a to indicated FcγR⁺ CHO transfectants pre-incubated or not with indicated mAbs (anti-hFcγRI.1, anti-hFcγRI.2, anti-FcγRIV). Solid gray histograms represent background fluorescence.

FIG. 8. Activity of the anti-FcγRIIA antibody IV.3. for treating thrombocytopenia in hFcγRIIA^{tg} mice. hFcγRIIA^{tg} FcRγ^{-/-} mice were pretreated with 50 μg IV.3 or not 24 h before being injected i.v. with anti-platelet mAb 6A6. Platelet counts were acquired in blood at t=4 hours following anti-platelet mAb injection (n=3).

FIG. 9. Activity of the anti-FcγRIIA antibody IV.3. for treating arthritis in the K/B×N inflammatory arthritis model in FcγRIIA-transgenic mice. (A-D) K/B×N passive arthritis in FcγRII^{tg} FcRγ^{-/-} mice (γ^{-/-} IIA) or in control FcRγ^{-/-} (γ^{-/-}) mice injected with arthritogenic serum from K/B×N mice (40-50 μL of K/B×N serum/mouse on day 0). (A) hFcγRIIA^{tg} FcRγ^{-/-} mice (γ^{-/-} IIA) or FcRγ^{-/-} mice (γ^{-/-}) were injected with K/B×N serum or PBS. (B) Anti-TNF-α blocking mAbs (from eBiosciences, 30 μg/mouse) were injected on day 0 to FcγRII^{tg} FcRγ^{-/-} mice (γ^{-/-} IIA) mice. (C) Anti-FcγRIIA mAbs (IV.3, 20 μg/mouse), but not isotype controls (IgG control, 20 μg/mouse) injected on days −1/0/+1/+2/+4 abolish K/B×N arthritis in FcγRII^{tg} FcRγ^{-/-} mice (γ^{-/-} IIA). (D) Human intravenous immunoglobulins (IVIG: gammunex, 1 g/kg)) were injected on day 0 to FcγRII^{tg} FcRγ^{-/-} mice (γ^{-/-} IIA) or FcRγ^{-/-} mice (γ^{-/-}).

### DEFINITIONS

The term “antibody” as used herein designates a protein that exhibit binding specificity to a specific antigen and often induces molecular or cellular responses. This term is intended to include monoclonal antibodies, polyclonal antibodies, and chimeric antibodies. More particularly, an antibody (or “immunoglobulin”) consists of a glycoprotein comprising at least two heavy (H) chains and two light (L) chains inter-connected by disulfide bonds.

Each heavy chain comprises a heavy chain variable region (or domain) (abbreviated herein as V_{H}) and a heavy chain constant region (hereafter C_{H}). Heavy chains are classified as gamma, mu, alpha, delta, or epsilon, and define the antibody's isotype as IgG, IgM, IgA, IgD, and IgE, respectively. The C_{H} region of the immunoglobulin IgG, IgD, and IgA (γ, δ and α chains respectively) comprises three domains (CH1, CH2, and CH3) and a hinge region for added flexibility, while the C_{H} region of the immunoglobulin IgM and IgE contains 4 domains (CH1, CH2, CH3, and CH4).

IgG antibodies are classified in four distinct subtypes, named IgG1, IgG2, IgG3 and IgG4. The structure of the hinge regions in the γ chain gives each of these subtypes its unique biological profile (even though there is about 95% similarity between their Fc regions, the structure of the hinge regions is relatively different).

Each light chain comprises a light chain variable region (abbreviated herein as V_{L}) and a light chain constant region comprising only one domain, C_{L}. There are two types of light chain in mammals: the kappa (K) chain, encoded by the immunoglobulin kappa locus on chromosome 2, and the lambda (A) chain, encoded by the immunoglobulin lambda locus on chromosome 22.

The V_{H} and V_{L} regions can be further subdivided into regions of hypervariability, termed “Complementarity Determining Regions” (CDR), which are primarily responsible for binding an antigen, and which are interspersed with regions that are more conserved, designated “Framework Regions” (FR). Each V_{H} and V_{L} is composed of three CDRs and four FRs, arranged from amino-terminus to carboxy-terminus in the following order: FR1, CDR1, FR2, CDR2, FR3, CDR3, FR4. The assignment of amino acid sequences to each domain is in accordance with well-known conventions (for example, the IMGT unique numbering convention as disclosed by Lefranc, M.-P., et al., Dev. Comp. Immunol., 27, 55-77 (2003)). The functional ability of the antibody to bind a particular antigen depends on the variable regions of each light/heavy chain pair, and is largely determined by the CDRs. The variable region of the heavy chain differs in antibodies produced by different B cells, but is the same for all antibodies produced by a single B cell or B cell clone (or hybridome). By contrast, the constant regions of the antibodies mediate the binding of the immunoglobulin to host tissues or factors, including various cells of the immune system (e.g. effector cells) and the first component (C1q) of the classical complement system.

An “epitope” is the site on the antigen to which an antibody binds. It can be formed by contiguous residues or by non-contiguous residues brought into close proximity by the folding of an antigenic protein.

A “polyclonal antibody” as used herein, designates an antibody that is obtained from different B cells. It typically includes various antibodies directed against various determinants, or epitopes, of the target antigen. These antibodies may be produced in animals by conventional techniques that are fully explained in the literature. For example, polyclonal antibodies may be prepared by immunizing a mammal (e.g. a mouse, hamster, or rabbit) with an immunogenic form of the antigen, which elicits an antibody response in the mammal. Following immunization, antisera can be obtained and, if desired, polyclonal antibodies isolated from the sera by conventional means.

A “monoclonal antibody”, as used herein, means an antibody arising from a nearly homogeneous antibody population. The individual antibodies of a population are identical except for a few possible naturally-occurring mutations which can be found in minimal proportions. In other words, a monoclonal antibody consists of a homogeneous antibody arising from the growth of a single cell clone (for example a hybridoma, a eukaryotic host cell transfected with a DNA molecule coding for the homogeneous antibody, a prokaryotic host cell transfected with a DNA molecule coding for the homogeneous antibody, etc.) and is characterized by heavy chains of one and only one isotype and subtype, and light chains of only one type. Monoclonal antibodies are highly specific and are directed against a single epitope of an antigen.

A “chimeric antibody”, as used herein, is an antibody in which the constant region, or a portion thereof, is altered, replaced, or exchanged, so that the variable region is linked to a constant region of a different species, or belonging to another antibody class or subclass. “Chimeric antibody” also refers to an antibody in which the variable region, or a portion thereof, is altered, replaced, or exchanged, so that the constant region is linked to a variable region of a different species, or belonging to another antibody class or subclass.

As used herein, the term “humanized antibody” refers to a chimeric antibody which contains minimal sequence derived from non-human immunoglobulin. It refers to an antibody that comprises CDR regions derived from an antibody of non-human origin, the other parts of the antibody molecule being of human origin. These antibodies are less immunogenic for human than the chimeric ones.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues.

As used herein, the term “antibody fragments” intends to designate Fab, Fab′, F(ab′)2, scFv, dsFv, ds-scFv, dimers, minibodies, diabodies, and multimers thereof and bispecific antibody fragments. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

A “functional fragment” of an antibody means in particular an antibody fragment as defined above, with the same binding and blocking activity to hFcγRI as the parental antibody.

It must be understood here that the invention does not relate to the antibodies in their natural form (that is to say “in their natural environment”) but that they have been isolated or obtained by purification from natural sources, by genetic recombination, or by chemical synthesis.

In the context of the present invention, an antibody is said to “recognize” or “bind” a receptor, for instance the hFcγRI receptor or the hFcγRIIA receptor, if said antibody has an affinity constant Kₐ (which is the inverted dissociation constant, i.e. 1/K_{d}) higher than 10⁷ M⁻¹, preferably higher than 10⁸ M⁻¹, more preferably higher than 10⁹ M⁻¹ for said receptor. Also, in the context of the present invention, an antibody is said to “specifically bind” or to “specifically recognize” a receptor if said antibody has an affinity constant Kₐ higher than 10⁷ M⁻¹, preferably higher than 10⁸ M⁻¹, more preferably higher than 10⁹ M⁻¹ for said receptor and has an affinity constant Kₐ that is at least two-fold less for other peptides, for example BSA or casein, especially for the other human Fcγreceptors (FcγRIIA, FcγRIII, etc.).

The affinity constant which is used to characterize the binding of antibodies (Ab) to a peptide or an antigen (Ag) is the inverted dissociation constant defined as follows:

$$Ab + Ag ⇌ AbAg$$

$${K}_{a} = \frac{[ AbAg ]}{[ Ab ] [ Ag ]} = \frac{1}{{K}_{d}}$$

This affinity can be measured for example by equilibrium dialysis or by fluorescence quenching, both technologies being routinely used in the art. It is also possible to use Biacore analysis to measure this affinity.

In the context of the present invention, a “blocking antibody” is an antibody that does not have or trigger a reaction when binding an antigen, but prevents at least one other ligand from binding to the antigen. More specifically, an antibody is said to “block” the hFcγRI or the hFcγRIIA receptor if it is capable of inhibiting the binding of said receptor with all natural ligand(s) thereof (IgG1, IgG2, IgG3 and/or IgG4) upon binding of the said antibody to the receptor. Standard assays to evaluate the binding ability of the antibodies toward the hFcγRI or the hFcγRIIA receptor are known in the art, including for example ELISAs, Western Blots and RIAs. A suitable assay is described in the Examples. By impairing the binding of the hFcγRI receptor or the hFcγRIIA receptor with their ligands, a blocking antibody also inhibits the activation of said receptor by these ligands. Alternatively, a blocking antibody may not impair the binding of the natural ligands onto the receptor, but may rather impair the signaling pathway induced by said binding. The blocking capacity of said antibody will be assessed by measuring the activation level of the cell expressing the receptors, for example, by measuring the phosphorylation status of the ITAM(s) known to be activated by said receptors, by any conventional means (for example by Western Blot). The blocking capacity of an antibody towards the hFcγRI and/or hFcγRIIA receptor(s) can be for example evaluated by measuring the inhibition of monomeric IgG and/or IgG-immune complex binding to hFcγRI or hFcγRIIA expressed by transfectants such as hFcγRI-expressing CHO cells (CNCM I-4383), hFcγRIIA(H131 isoform)-expressing CHO cells (CNCM I-4384) and hFcγRIIA(R131 isoform)-expressing CHO cells (CNCM I-4385). Binding conditions as described in Bruhns P et al., Blood 2009; 113:3716-3725. As used herein, “glycosylation pattern” is defined as the pattern of carbohydrate units that are covalently attached to a protein, more specifically to an immunoglobulin protein. A glycosylation pattern of a chimeric antibody can be characterized as being substantially similar to glycosylation patterns which occur naturally on antibodies produced by the species of the nonhuman transgenic animal.

The terms “nucleic acid”, “nucleic sequence”, “nucleic acid sequence”, “polynucleotide”, “oligonucleotide”, “polynucleotide sequence”, used interchangeably in the present description, mean a sequence of nucleotides, modified or not, defining a fragment or a region of a nucleic acid, containing unnatural nucleotides or not, and being either a double-strand DNA, a single-strand DNA or transcription products of said DNAs (mRNA).

Importantly, the present invention does not relate to nucleotide sequences in their natural chromosomal environment, i.e., in a natural state. The sequences of the present invention have been isolated and/or purified, i.e., they were sampled directly or indirectly, for example by a copy, their environment having been at least partially modified. Isolated nucleic acids obtained by recombinant genetics, by means, for example, of host cells, or obtained by chemical synthesis should also be mentioned here.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e. g., bacterial vectors having a bacterial origin of replication and episomal mammalian vectors). Other vectors (e. g., non-episomal mammalian vectors) can be integrated into the genome of a host cell upon introduction into the host cell, and thereby are replicated along with the host genome. Certain vectors are capable of directing the expression of genes to which they are operatively linked. Such vectors are referred to herein as “recombinant expression vectors” (or simply, “expression vectors”). In general, expression vectors of utility in recombinant DNA techniques are in the form of plasmids. In the present specification, “plasmid” and “vector” may be used interchangeably as the plasmid is the most commonly used form of vector. However, the invention is intended to include such forms of expression vectors, such as bacterial plasmids, YACs, cosmids, retrovirus, EBV-derived episomes, and all the other vectors that the skilled man will know to be convenient for ensuring the expression of the heavy and/or light chains of the antibodies of the invention.

The term “host cell”, as used herein, is intended to refer to a cell into which a recombinant expression vector has been introduced in order to express the antibody of the invention. It should be understood that such terms are intended to refer not only to the particular subject cell but also to the progeny of such a cell. Because certain modifications may occur in succeeding generations due to either mutation or environmental influences, such progeny may not, in fact, be identical to the parent cell, but are still included within the scope of the term “host cell” as used herein. In addition, a host cell is chosen which modulates the expression of the inserted sequences, or modifies and processes the gene product in the specific fashion desired. Such modifications (e.g., glycosylation) and processing of protein products may be important for the function of the protein. Different host cells have features and specific mechanisms for the post-translational processing and modification of proteins and gene products. Appropriate cell lines or host systems are chosen to ensure the correct modification and processing of the expressed antibody of interest. Hence, eukaryotic host cells (and in particular mammalian host cells) which possess the cellular machinery for proper processing of the primary transcript, glycosylation of the gene product may be used. Such mammalian host cells include, but are not limited to, Chinese hamster cells (e.g. CHO cells), monkey cells (e.g. COS cells), human cells (e.g. HEK293 cells), baby hamster cells (e.g. BHK cells), NS/0, Y2/0, 3T3 or myeloma cells (all these cell lines are available from public depositories such as the Collection Nationale des Cultures de Microorganismes, Paris, France, or at the American Type Culture Collection, Manassas, Va., U.S.A.). Alternatively, the yeast cell may be a yeast cell that has been engineered so that the glycosylation (and in particular N-glucosylation) mechanisms are similar or identical to those taking place in a mammalian cell. For long-term, high-yield production of recombinant proteins, stable expression is preferred. Mammalian cells are commonly used for the expression of recombinant therapeutic immunoglobulins, especially for the expression of whole recombinant IgG antibodies.

“Operably linked” sequences include both expression control sequences that are contiguous with the gene of interest and expression control sequences that act in trans or at a distance to control the gene of interest. The term “expression control sequence” as used herein refers to polynucleotide sequences which are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i. e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

In the present description, “pharmaceutically acceptable carrier” means a compound, or a combination of compounds, contained in a pharmaceutical composition, that does not cause secondary reactions and that, for example, facilitates administration of the active compounds, increases its lifespan and/or effectiveness in the organism, increases its solubility in solution or improves its storage. Such pharmaceutical carriers are well-known and will be adapted by a person skilled in the art according to the nature and the administration route of the active compounds selected. A typical pharmaceutical composition for intravenous infusion could be made up to contain 250 ml of sterile Ringer's solution, and 100 mg of the combination. Actual methods for preparing parenterally administrable compounds will be known or apparent to those skilled in the art and are described in more detail in for example, Remington's Pharmaceutical Science, 17th ed., Mack Publishing Company, Easton, Pa. (1985), and the 18th and 19th editions thereof, which are incorporated herein by reference.

As used herein, the term “subject” designates an individual of any animal species, including mammals and more precisely human. Preferably, it is a human.

### DETAILED DESCRIPTION OF THE INVENTION

The Antibody Anti-FcγRI.1. Of the Invention

Structure of the Antibody of the Invention

In a first aspect, the present invention relates to an isolated antibody or a functional fragment thereof, that binds and blocks the human FcγRI receptor, preferably of SEQ ID NO:7, said antibody or fragment comprising at least one, preferably two, preferably three, preferably four, preferably five and more preferably six CDR(s) consisting of SEQ ID NO:1-6 of the enclosed sequence listing.

In a preferred embodiment, said antibody or fragment comprises the CDR(s) consisting of SEQ ID NO:1-6 of the enclosed sequence listing.

In a more preferred embodiment, said antibody or fragment comprises a heavy chain comprising three CDRs having the following amino acid sequences:

(SEQ ID NO: 1)
i) CDR1: GFSLTTYG;
(SEQ ID NO: 2)
ii) CDR2: IWSGGST;
(SEQ ID NO: 3)
iii) CDR3: AREWFAY.

In a more preferred embodiment, said antibody or fragment comprises a light chain comprising three CDRs having the following amino acid sequences:

(SEQ ID NO: 4)
i) CDR1: ENIYSY;
(SEQ ID NO: 5)
ii) CDR2: SAK;
(SEQ ID NO: 6)
iii) CDR3: QHHYGTPYT.

In an even more preferred embodiment, the antibody or fragment of the invention comprises:
a) a heavy chain comprising three CDRs having the following amino acid sequences:

(SEQ ID NO: 1)
i) the heavy chain CDR1: GFSLTTYG;
(SEQ ID NO: 2)
ii) the heavy chain CDR2: IWSGGST;
(SEQ ID NO: 3)
iii) the heavy chain CDR3: AREWFAY,

and
b) a light chain comprising three CDRs having the following amino acid sequences:

(SEQ ID NO: 4)
i) the light chain CDR1: ENIYSY;
(SEQ ID NO: 5)
ii) the light chain CDR2: SAK;
(SEQ ID NO: 6)
iii) the light chain CDR3: QHHYGTPYT.

The skilled person easily understands that the present invention also relates to antibodies or fragments whose CDRs are not strictly identical to SEQ ID NO:1-6. The CDRs of these antibodies or fragments can contain conservative modifications, i.e., amino acid sequence modifications which do not significantly affect or alter the binding characteristics of the antibody containing the amino acid sequence. Such conservative sequence modifications include amino acid substitutions, additions and deletions. Modifications can be introduced into SEQ ID NOs: 1-6 and/or 11-16 by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e. g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in a human anti-hFcγRI antibody is preferably replaced with another amino acid residue from the same side chain family.

In a preferred embodiment, the antibody or fragment of the invention comprises a heavy chain variable region (V_{H}) having the amino acid sequence SEQ ID NO: 9.

In a more preferred embodiment, the antibody or fragment of the invention comprises a heavy chain variable region (V_{H}) having the amino acid sequence SEQ ID NO: 9, and a light chain comprising three CDRs having the following amino acid sequences:

(SEQ ID NO: 4)
i) the light chain CDR1: ENIYSY;
(SEQ ID NO: 5)
ii) the light chain CDR2: SAK;
(SEQ ID NO: 6)
iii) the light chain CDR3: QHHYGTPYT.

In another preferred embodiment, the antibody or fragment of the invention comprises a light chain variable region (V_{L}) having the amino acid sequence SEQ ID NO: 10.

In a more preferred embodiment, the antibody or fragment of the invention comprises a light chain variable region (V_{L}) having the amino acid sequence SEQ ID NO: 10, and a heavy chain comprising three CDRs having the following amino acid sequences:

(SEQ ID NO: 1)
i) CDR1: GFSLTTYG;
(SEQ ID NO: 2)
ii) CDR2: IWSGGST;
(SEQ ID NO: 3)
iii) CDR3: AREWFAY.

In another preferred embodiment, the antibody or fragment of the invention comprises a heavy chain variable region (V_{H}) having the amino acid sequence SEQ ID NO: 9 and a light chain variable region (V_{L}) having the amino acid sequence SEQ ID NO: 10.

The antibody of the invention can be of the IgG, IgM, IgA, IgD, and IgE isotype, depending on the structure of its heavy chain. However, in a preferred embodiment, the antibody of the invention is of the IgG isotype, i.e., its heavy chain is of the gamma (γ) type.

Non-Immunogenicity of the Antibody of the Invention

The Fc domains are central in determining the biological functions of the immunoglobulin and these biological functions are termed “effector functions”. These Fc domain-mediated activities are mediated via immunological effector cells, such as killer cells, natural killer cells, and activated macrophages, or various complement components. These effector functions involve activation of receptors on the surface of said effector cells, through the binding of the Fc domain of an antibody to the said receptor or to complement component(s). The antibody-dependent cellular cytotoxicity (ADCC) and complement-dependent cytotoxicity (CDC) activities involve the binding of the Fc domain to Fc-receptors such as FcγRI, FcγRII, FcγRIII of the effector cells or complement components such as C1q. Of the various human immunoglobulin classes, human IgG1 and IgG3 mediate ADCC more effectively than IgG2 and IgG4.

The antibody of the invention can be of the IgG1, IgG2, IgG3 or IgG4 subtype. However, in a preferred embodiment, the antibody of the invention is of the IgG1 subtype. Preferably, in this case, it contains one mutation (N₂₉₇D in the constant region of the human IgG1 heavy chain) abolishing the binding to other Fc Receptors. Consequently, the antibody does not exhibit effector functions, such as antibody-dependent cellular cytotoxicity (ADCC).

The antibodies of the invention may also contain any mutations that prevent effector functions such as antibody-dependent cellular cytotoxicity (ADCC) and/or complement-dependent cytotoxicity (CDC). These mutations are well-known in the art. They are described for example in WO 2004/029207, WO 2000/42072, WO 2007/106707, WO 2007/024249, WO 2007/021841, WO 2008/002933 and in Stavenhagen et al, Cancer Res. 2007; 67; 18; p.8882-8890).

Finally, the antibodies of the invention have preferably a Fc portion that does not present a correct glycosylation able to confer effector functions (for example, due to the N₂₉₇D mutation in the human constant regions of the IgG1 heavy-chain).

Nature of the Antibody of the Invention

The antibody of the invention can be a monoclonal antibody or a polyclonal antibody. Preferably, it is a monoclonal antibody.

The monoclonal antibody of the invention can be from any origin: for example, the said monoclonal antibody can be a murine antibody. However the present invention is not restricted to monoclonal antibodies of murine origin. Actually, chimeric antibodies, humanized antibodies and human antibodies are also included within the scope of this invention. The goal of humanization is a reduction in the immunogenicity of a xenogenic antibody, such as a murine antibody, for introduction into a human, while maintaining the full antigen binding affinity and specificity of the antibody.

Preferably, the chimeric antibody of the invention comprises a variable region of the light chain and/or heavy chain that is derived from the anti-FcγRI.1 antibody, which is fused with constant regions of the light chain and the heavy chain of a human antibody. In a preferred embodiment, the heavy chain variable region of the chimeric antibody of the invention has the sequence SEQ ID NO:19. In another preferred embodiment, the light chain variable region of the chimeric antibody of the invention has the sequence SEQ ID NO:20. In a more preferred embodiment, the chimeric antibody of the invention has an heavy chain variable region of sequence SEQ ID NO:19 and a light chain variable region of sequence SEQ ID NO:20.

In a more preferred embodiment, the said monoclonal antibody is a humanized antibody, for example containing the CDR regions of mouse anti-FcγRI.1, the other parts of the antibody molecule being of human origin.

Preferably, the humanized antibody of the invention comprises a heavy chain variable region from a particular germline heavy chain immunoglobulin gene and/or a light chain variable region from a particular germline light chain immunoglobulin gene.

More preferably, the humanized anti-hFcγRI antibody of the invention has a heavy chain variable region comprising 3 CDRs having the sequences SEQ ID NO: 1, 2 and 3.

More preferably, the humanized anti-hFcγRI antibody of the invention has a light chain variable region comprising 3 CDRs having the sequences SEQ ID NO: 4, 5 and 6.

Even more preferably, the humanized anti-hFcγRI antibody of the invention has a heavy chain variable region comprising 3 CDRs having the sequences SEQ ID NO: 1, 2 and 3 and a light chain variable region comprising 3 CDRs having the sequences SEQ ID NO: 4, 5 and 6.

In another embodiment, the invention relates to a human anti-hFcγRI antibody or functional fragment of same, said antibody having a heavy chain variable region comprising three CDRs having the sequences SEQ ID NO: 1, 2 and 3, and/or a light chain variable region comprising three CDRs having the sequences SEQ ID NO:4, 5 and 6.

Production of the Antibody of the Invention

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e. g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups. Animals are immunized against the antigen, immunogenic conjugates, or derivatives by combining, e.g., 100 pg or 5 pg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal antibodies may be made using the hybridoma method first described by Kohler et al., Nature, 256: 495 (1975), or may be made by recombinant DNA methods (U.S. Pat. No. 4,816,567). In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells. Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133: 3001 (1984); Brodeur et al., Monoclonal Antibody Production Techniques and Applications, pp. 51-63 (Marcel Dekker, Inc., New York, 1987)). Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). After hybridoma cells are identified that produce antibodies of the desired specificity, affinity, and/or activity, the clones may be subcloned by limiting dilution procedures and grown by standard methods (Goding, Mofzoclotzal Afztibodies: Principles and Practice, pp. 59-103 (Academic Press, 1988)). Suitable culture media for this purpose include, for example, D-MEM or RPMI-1640 medium. In addition, the hybridoma cells may be grown in vivo as ascites tumors in an animal. The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography. In a further embodiment, antibodies or antibody fragments can be isolated from antibody phage libraries generated using the techniques described in McCafferty et al., Nature, 348: 552-554 (1990). Clackson et al., Nature, 352: 624-628 (1991) and Marks et al., J. Mol. Biol., 222: 581-597 (1991) describe the isolation of murine and human antibodies, respectively, using phage libraries. Subsequent publications describe the production of high affinity (nM range) human antibodies by chain shuffling (Marks et al., Biotechnology, 10: 779-783 (1992)), as well as combinatorial infection and in vivo recombination as a strategy for constructing very large phage libraries (Waterhouse et al., Nuc. Acid. Res., 21: 2265-2266 (1993)). Thus, these techniques are viable alternatives to traditional monoclonal antibody hybridoma techniques for isolation of monoclonal antibodies.

Humanized antibody may be produced using several technologies such as resurfacing and CDR grafting. As used herein, the resurfacing technology uses a combination of molecular modeling, statistical analysis and mutagenesis to alter the non-CDR surfaces of antibody variable regions to resemble the surfaces of known antibodies of the target host. Strategies and methods for the resurfacing of antibodies, and other methods for reducing immunogenicity of antibodies within a different host, are disclosed in U.S. Pat. No. 5,639,641. Another method of humanization of antibodies, based on the identification of flexible residues, has been described in PCT application WO 2009/032661. Antibodies can be humanized using a variety of other techniques including CDR-grafting (EP 0 239 400; WO 91/09967; U.S. Pat. No. 5,530,101; and U.S. Pat. No. 5,585,089), veneering or resurfacing (EP 0 592 106; EP 0 519 596; Padlan E. A., 1991, Mol Immunol, 28(4/5): 489-498; Studnicka G. M. et al., 1994, Protein Engineering 7(6): 805-814; Roguska M. A. et al., 1994, Proc. Natl. Acad. Sci. U.S.A., 91: 969-973), and chain shuffling (U.S. Pat. No. 5,565,332).

Human antibodies can be produced using various techniques known in the art. In one embodiment, the human antibody is selected from a phage library, where that phage library expresses human antibodies (Vaughan et al. Nature Biotechnology 14: 309-314 (1996): Sheets et al. PNAS (USA) 95: 6157-6162 (1998)); Hoogenboom and Winter, J. Mol. Biol., 227: 381 (1991); Marks et al., J. Mol. Biol., 222: 581 (1991)). Human antibodies can also be made by introducing human immunoglobulin loci into transgenic animals, e. g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in Lonberg and Huszar, Intern. Rev. Immunol. 13: 65-93 (1995). Alternatively, the human antibody may be prepared via immortalization of human B lymphocytes producing an antibody directed against a target antigen (such B lymphocytes may be recovered from an individual or may have been immunized in vitro).

Nucleic Acids Encoding the Antibody of the Invention

In another aspect, the present invention also relates to an isolated nucleic acid selected among the following nucleic acids:
a) a nucleic acid, DNA or RNA, coding for a mouse antibody heavy chain containing SEQ ID NO:1, SEQ ID NO:2 and/or SEQ ID NO:3,
b) a nucleic acid, DNA or RNA, coding for a mouse antibody light chain containing SEQ ID NO:4, SEQ ID NO:5 and/or SEQ ID NO:6,
c) a nucleic acid, DNA or RNA, coding for a mouse antibody heavy chain and a mouse light chain containing SEQ ID NO:1, SEQ ID NO:2, SEQ ID NO:3, SEQ ID NO:4, SEQ ID NO:5 and/or SEQ ID NO:6,
d) a nucleic acid, DNA or RNA, coding for a chimeric antibody heavy chain containing SEQ ID NO:1, SEQ ID NO:2 and/or SEQ ID NO:3, said heavy chain having preferably the sequence SEQ ID NO: 19;
e) a nucleic acid, DNA or RNA, coding for a chimeric antibody light chain containing SEQ ID NO:4, SEQ ID NO:5 and/or SEQ ID NO:6, said light chain having preferably the sequence SEQ ID NO: 20,
f) a nucleic acid, DNA or RNA, coding for a chimeric heavy chain and a chimeric light chain containing SEQ ID NO:1, SEQ ID NO:2, SEQ ID NO:3, SEQ ID NO:4, SEQ ID NO:5 and/or SEQ ID NO:6,
g) a nucleic acid complementary to a nucleic acid as defined in a), b), c), d), e) or f);

In a preferred embodiment, said nucleic acid codes for a murine heavy chain having the sequence SEQ ID NO:9. In another preferred embodiment, said nucleic acid codes for a murine light chain having the sequence SEQ ID NO:10. In another preferred embodiment, said nucleic acid codes for a murine heavy chain having the sequence SEQ ID NO:9 and a murine light chain having the sequence SEQ ID NO:10.

In another preferred embodiment, said nucleic acid codes for a humanized heavy chain having the sequence SEQ ID NO: 19. In another preferred embodiment, said nucleic acid codes for a light chain having the sequence SEQ ID NO: 20. In another preferred embodiment, said nucleic acid codes for a humanized heavy chain having the sequence SEQ ID NO: 19 and a light chain having the sequence SEQ ID NO: 20.

Said nucleic acid has preferably the sequence chosen in the group consisting of: SEQ ID NO:28 (corresponding to the nucleic acid defined in a), SEQ ID NO: 29 (corresponding to the nucleic acid defined in b), SEQ ID NO:23 (corresponding to the nucleic acid defined in d) and SEQ ID NO:24 (corresponding to the nucleic acid defined in e). The present invention also concerns any polynucleotide whose sequence is homologous to SEQ ID NO:23, 24, 28 and/or 29 but, due to codon degeneracy, does not contain precisely the same nucleotide sequence.

Vectors of the Invention

The invention also provides vectors comprising the polynucleotides of the invention. In one embodiment, the vector contains a polynucleotide encoding a heavy chain of the antibody of the invention. In another embodiment, said polynucleotide encodes the light chain of the antibody of the invention. The invention also provides vectors comprising polynucleotide molecules encoding fusion proteins, modified antibodies, or antibody fragments thereof.

More precisely, the present invention relates to an expression vector containing at least one of nucleic acid sequence a) to g) described above. In a preferred embodiment, said vector is a viral vector or a plasmid or a naked DNA. In order to efficiently express the heavy and/or light chain of the antibody of the invention, the polynucleotides encoding said heavy and/or light chains or fragments thereof are operatively linked to transcriptional and translational sequences that are present in said expression vectors.

The skilled man will realize that the polynucleotides encoding the heavy and the light chains can be cloned into different vectors or in the same vector. In one embodiment, said polynucleotides are cloned into two vectors.

Polynucleotides of the invention and vectors comprising these molecules can be used for the transformation of a suitable host cell. Transformation of host cells can be performed by any known method for introducing polynucleotides into a cell host. Such methods are well known of the man skilled in the art and include dextran-mediated transformation, calcium phosphate precipitation, polybrene-mediated transfection, protoplast fusion, electroporation, encapsulation of the polynucleotide into liposomes, biolistic injection and direct microinjection of DNA into nuclei. The host cell may be co-transfected with two or more expression vectors, including the vector expressing the protein of the invention.

Host Cells of the Invention

In another aspect, the present invention therefore relates to a host cell containing the expression vector of the invention and therefore expressing the antibody of the invention or a functional fragment thereof. This host cell can be any cell, provided that it is not a human embryonic stem cell or a human germinal cell.

In a preferred embodiment, the host cell of the invention is a mammalian cell. More preferably, it is a HEK 293T cell. In this case, a suitable promoter that can be used in the vector of the invention is for example the T7 promoter or the human cytomegalovirus early promoter (CMV). Preferably, the vectors of the invention contain an ampicillin selectable marker and SV40, ColEI and fl origin of replication.

In one embodiment of the invention, cell lines which stably express the antibody of the invention may be engineered. Using expression vectors which contain viral origins of replication, host cells can be transformed with DNA under the control of the appropriate expression regulatory elements and a selectable marker. Following the introduction of the foreign DNA, engineered cells may be allowed to grow for one to two days in an enriched media, and then are moved to a selective media. The selectable marker on the recombinant plasmid confers resistance to the selection and allows cells to stably integrate the plasmid into a chromosome and be expanded into a cell line. Other methods for constructing stable cell lines are known in the art. In particular, methods for site-specific integration have been developed. According to these methods, the transformed DNA under the control of the appropriate expression regulatory elements is integrated in the host cell genome at a specific target site which has previously been cleaved (U.S. Pat. No. 5,792,632; U.S. Pat. No. 5,830,729; U.S. Pat. No. 6,238,924; WO 2009/054985; WO 03/025183; WO 2004/067753). A number of selection systems may be used according to the invention, including but not limited to the Herpes simplex virus thymidine kinase (Wigler et al., Cell 1 1:223, 1977), hypoxanthine-guanine phosphoribosyltransferase (Szybalska et al., Proc Natl Acad Sci USA 48: 202, 1992), glutamate synthase selection in the presence of methionine sulfoximide (Adv Drug Del Rev, 58: 671, 2006, and website or literature of Lonza Group Ltd.) and adenine phosphoribosyltransferase (Lowy et al., Cell 22: 817, 1980) genes in tk, hgprt or aprt cells, respectively. Also, antimetabolite resistance can be used as the basis of selection for the following genes: dhfr, which confers resistance to methotrexate (Wigler et al., Proc Natl Acad Sci USA 77: 357, 1980); gpt, which confers resistance to mycophenolic acid (Mulligan et al., Proc Natl Acad Sci USA 78: 2072, 1981); neo, which confers resistance to the aminoglycoside, G-418 (Wu et al., Biotherapy 3: 87, 1991); and hygro, which confers resistance to hygromycin (Santerre et al., Gene 30: 147, 1984). Methods known in the art of recombinant DNA technology may be routinely applied to select the desired recombinant clone, and such methods are described, for example, in Ausubel et al., eds., Current Protocols in Molecular Biology, John Wiley & Sons (1993). The expression levels of an antibody can be increased by vector amplification. When a marker in the vector system expressing an antibody is amplifiable, an increase in the level of inhibitor present in the culture will increase the number of copies of the marker gene. Since the amplified region is associated with the gene encoding the antibody of the invention, production of said antibody will also increase (Crouse et al., Mol Cell Biol 3: 257, 1983). Alternative methods of expressing the polynucleotides of the invention exist and are known to the person of skills in the art.

The antibody of the invention may be prepared by growing a culture of the transformed host cells under culture conditions necessary to express the desired antibody. The resulting expressed antibody may then be purified from the culture medium or cell extracts. Soluble forms of the antibody of the invention can be recovered from the culture supernatant. It may then be purified by any method known in the art for purification of an immunoglobulin molecule, for example, by chromatography (e.g., ion exchange, affinity, particularly by Protein A affinity for Fc, and so on), centrifugation, differential solubility or by any other standard technique for the purification of proteins. Suitable methods of purification will be apparent to a person of ordinary skills in the art.

Therapeutical Uses of the Antibody of the Invention

Inflammation is characterized by increased blood flow, increased capillary permeability, and the influx of phagocytic cells. These events result in swelling, redness, warmth (altered heat patterns), and pus formation at the site of injury or infection.

As shown in the experimental part below, the present inventors report here that hFcγRI induces several mouse models of auto-immune and allergic reactions, and that the antibody of the invention (anti-hFcγRI.1) prevented and/or abolished the symptoms of these reactions. More specifically, blocking the hFcγRI receptor with the monoclonal antibody anti-hFcγRI.1 allows to significantly reduce i) the arthritis symptoms, ii) antibody-dependent airway inflammation, iii) passive and active systemic anaphylaxis and iv) thrombocytopenia in transgenic mice suffering therefrom.

The humanized antibodies of the invention may thus be very useful for treating inflammation-related human pathologies such as airway inflammation, systemic anaphylaxis, autoimmune arthritis and thrombocytopenia.

The present invention therefore relates to a pharmaceutical composition (or a medicament) comprising, as an active ingredient, an efficient amount of the antibody of the invention, or one of its functional fragments. Preferably, said antibody (or fragment) is supplemented by an excipient and/or a pharmaceutically acceptable carrier.

An “effective amount” refers to an amount effective, at dosages and for periods of time necessary, to achieve the desired result, such as prevention or treatment of the diseases mentioned above.

More precisely, the present invention relates to the antibody of the invention, a functional fragment thereof, or the pharmaceutical composition of the invention, for use for preventing and/or treating IgG antibody-dependent inflammatory and autoimmune disorders. These disorders are typically arthritis, related arthritic conditions (e.g., osteoarthritis, rheumatoid arthritis, and psoriatic arthritis), inflammatory bowel disease (e.g., Crohn's disease and ulcerative colitis), lupus, antibody-nephritis, allergic reactions, psoriasis, atopic dermatitis, contact dermatitis, antibody-induced anemia, chronic obstructive pulmonary disease, and chronic inflammatory pulmonary diseases.

Preferably, these inflammatory and autoimmune disorders are chosen in the group consisting of: arthritic symptoms, allergic reactions, lupus or antibody-nephritis.

In a preferred embodiment, said inflammatory disorder is rheumatoid arthritis. Rheumatoid arthritis (RA) is a chronic, systemic inflammatory disorder that may affect many tissues and organs, but principally attacks flexible (synovial) joints (Aletaha D, et al. Ann Rheum Dis. 2010, 69(9):1580-1588). The pathology of the disease process often leads to the destruction of articular cartilage and ankylosis (fusion) of the joints. Rheumatoid arthritis can also produce diffuse inflammation in the lungs, in the pericardium, in the pleura and in the sclera, and also nodular lesions, most common in subcutaneous tissue.

In another preferred embodiment, said inflammatory disorder is anaphylaxis. Anaphylaxis is an allergic inflammation causing a number of symptoms including an itchy rash, throat swelling, edema, bronchospasm, low blood pressure, hypothermia and, ultimately, death. Grade 1 is characterized by cutaneous signs. Grade 2 is characterized by moderate cardiovascular (hypotension, tachycardia) or bronchial dysfunction that does not require a specific treatment. Grade 3 is characterized by dysfunction with vital threat that would not have recessed in the absence of symptomatic treatment (cutaneous signs may be absent in this context or appear only when an adequate perfusion pressure has been re-established. Grade 4 is characterized by cardiorespiratory arrest (Soar, J. et al. Resuscitation 77, 157-169 (2008). Common causes include insect bites/stings, foods, and medications. On a pathophysiologic level, anaphylaxis is caused by the release of mediators from certain types of white blood cells triggered either by immunologic or non-immunologic mechanisms.

The present invention also relates to the use of the antibody of the invention or a functional fragment thereof, as defined above, for the manufacture of a pharmaceutical composition intended to prevent and/or treat IgG antibody dependent inflammatory and autoimmune disorders, in subjects in need thereof.

In other words, the present invention pertains to a method for treating a subject suffering from IgG antibody dependent inflammatory and autoimmune disorders, comprising the administration of an efficient amount of the antibody of the invention, a functional fragment thereof, or the pharmaceutical composition of the invention.

Preferably, these inflammatory and autoimmune disorders are chosen in the group consisting of: arthritic symptoms, allergic reactions, lupus or antibody-nephritis.

Preferably, the pharmaceutical composition of the invention will be administered by systemic route, notably by intravenous, intramuscular, intradermal, intraperitoneal, subcutaneous or oral route. More preferably, the composition composed of the antibody of the invention will be administered in several doses that are spaced equally over time. Their administration routes, dosing schedules and optimal galenic forms can be determined according to the criteria generally taken into account when establishing a treatment suited to a patient such as, for example, the patient's age or body weight, the seriousness of his general state, his tolerance for the treatment and the side effects experienced.

As demonstrated in the Examples below, blocking the hFcγRI receptor with the monoclonal antibody anti-hFcγRI.1 of the invention enables to significantly reduce the platelet consumption in transgenic mice suffering from Immune Thrombocytopenic Purpura (ITP).

In another aspect, the present invention therefore relates to the antibody of the invention, a functional fragment thereof, or the pharmaceutical composition of the invention, for use for preventing and/or treating thrombocytopenia in a subject in need thereof.

In human, thrombocytopenia (or thrombopenia) is a relative decrease of platelets in blood. One common definition of thrombocytopenia is a platelet count below 50,000 per microlitre. The present invention also relates to the use of the antibody of the invention, or a functional fragment thereof, for the manufacture of a pharmaceutical composition intended to prevent and/or treat thrombocytopenia, in subjects in need thereof.

In other words, the present invention relates to a method for treating a subject suffering from thrombocytopenia, comprising the administration of an effective amount of the antibody of the invention, a functional fragment thereof, or the pharmaceutical composition of the invention.

Said subject is preferably a human.

Novel Therapeutical Use of the Antibody Anti-FcγRIIA IV.3.

Human FcγRIIA (or CD32A) is a surface receptor protein. FcγRIIA is the most widely expressed FcR in humans, and remarkably the only activating IgG receptor constitutively expressed by mast cells, basophils, neutrophils, and eosinophils. FcγRIIA possesses its own ITAM in its intracytoplasmic domain, and is not associated with the FcRγ-subunit. FcγRIIA binds all 4 human IgG subclasses, as well as mouse IgG1, IgG2a, and IgG2b subclasses. The aggregation of FcγRIIA by IgG-immune complexes or by IgG-opsonized cells or surfaces induces the phosphorylation of the FcγRIIA ITAM and downstream signaling; thus, once aggregated, FcγRIIA can induce phagocytosis, cell activation, cell degranulation, cytokine release, microbe killing and the activation of the respiratory burst. Natural ligands for Human FcγRIIA are: human IgG1, IgG2, IgG3 and IgG4. (Jonsson F, et al. Blood 2012; 119(11):2533-2544).

The human FcγRIIA receptor has the sequence SEQ ID NO:8 (variant H131) or the SEQ ID NO:27 (variant R131). The terminology “FcγRIIA” herein represents both variants of sequences SEQ ID NO:8 and 27.

The present inventors have previously demonstrated that human FcγRIIA is sufficient to induce active and passive systemic anaphylaxis, cutaneous anaphylaxis, and lung inflammation in FcγRIIA-transgenic mice (Jonsson F. et al, Blood 2012; 119(11):2533-2544).

The monoclonal antibody anti-hFcγRIIA known as “IV.3” efficiently blocks the human FcγRIIA receptor (Looney R J, et al. J Immunol. 1986; 136(5):1641-1647).

The present inventors also demonstrated that the monoclonal antibody anti-hFcγRIIA “IV.3” abolished anaphylaxis and lung inflammation in mice model (Jonsson F. et al, Blood 2012; 119(11):2533-2544).

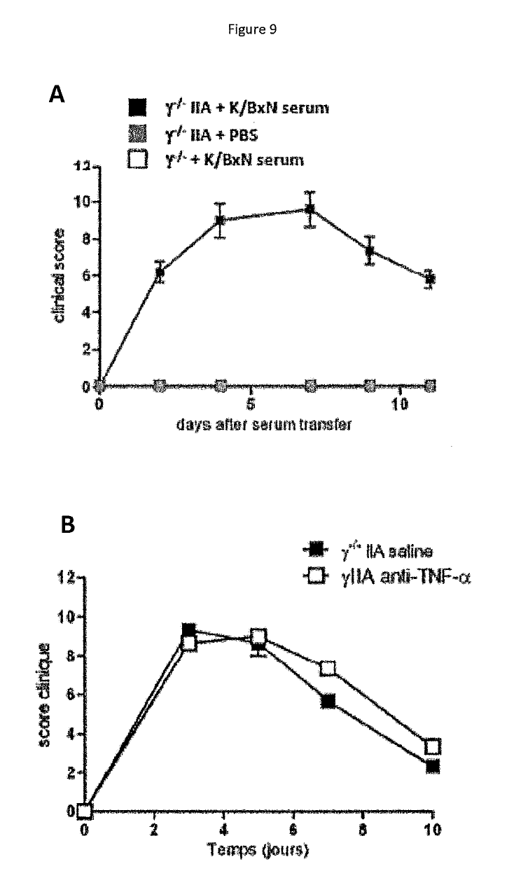

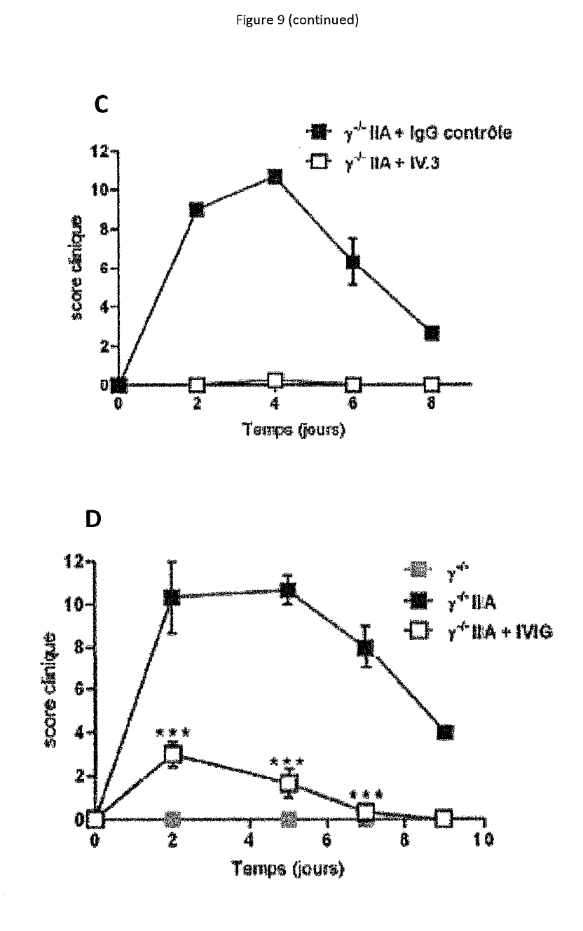

The present inventors have now demonstrated that the blocking antibody anti-hFcγRIIA IV.3 is also able to treat other inflammation-related disorders such as arthritis (see FIG. 9).

The antibody IV.3 is commercially available. This antibody comprises:
a) a heavy chain comprising three CDRs having the following amino acid sequences:

(SEQ ID NO: 11)
i) the heavy chain CDR1: GYTFTNYG;
(SEQ ID NO: 12)
ii) the heavy chain CDR2: LNTYTGES;
(SEQ ID NO: 13)
iii) the heavy chain CDR3: ARGDYGYDDPLDY,

and
b) a light chain comprising three CDRs having the following amino acid sequences:

(SEQ ID NO: 14)
i) the light chain CDR1: KSLLHTNGNTY;
(SEQ ID NO: 15)
ii) the light chain CDR2: RMSV;
(SEQ ID NO: 16)
iii) the light chain CDR3: MQHLEYPLT.

This antibody more precisely comprises a heavy chain variable region (V_{H}) having the amino acid sequence SEQ ID NO: 17 and/or a light chain variable region (V_{L}) having the amino acid sequence SEQ ID NO: 18.

The present inventors propose to use an antibody anti-hFcγRIIA IV.3 having at least one, preferably two, preferably three, preferably four, preferably five and even more preferably six CDR(s) of the antibody IV.3, or a functional fragment thereof, for preventing and/or treating inflammatory-related disorders in a subject in need thereof, preferably in human.

More precisely, they propose to use chimeric, humanized or human antibodies having at least one, preferably two, preferably three, preferably four, preferably five and even more preferably six CDR(s) of the antibody IV.3, or a functional fragment thereof, for preventing and/or treating inflammatory-related disorders in a subject in need thereof, preferably in human.

In a particular embodiment, the antibody which will be used in the treatment and prevention of said inflammatory-related disorders is a chimeric antibody (hereafter called a “chimeric form” of the said antibody) containing at least one, preferably two, preferably three, preferably four, preferably five and even more preferably six CDR(s) of the antibody IV.3 (SEQ ID NO:11 to 16), or a functional fragment thereof. More preferably, it is a chimeric antibody comprising a heavy chain variable region (V_{H}) having the amino acid sequence SEQ ID NO: 21 and/or a light chain variable region (V_{L}) having the amino acid sequence SEQ ID NO:22. In another particular embodiment, the antibody which will be used in the treatment and prevention of said inflammatory-related disorders is a humanized antibody (hereafter called a “humanized form” of the said antibody) containing at least one, preferably two, preferably three, preferably four, preferably five and even more preferably six CDR(s) of the antibody IV.3 (SEQ ID NO:11 to 16), or a functional fragment thereof. More preferably, it is a humanized antibody comprising a heavy chain variable region (V_{H}) having the amino acid sequence SEQ ID NO: 21 and/or a light chain variable region (V_{L}) having the amino acid sequence SEQ ID NO:22.

In another particular embodiment, the antibody which will be used in the treatment and prevention of said inflammatory-related disorders is a human antibody (hereafter called a “human form” of the said antibody) containing at least one, preferably two, preferably three, preferably four, preferably five and even more preferably six CDR(s) of the antibody IV.3 (SEQ ID NO:11 to 16), or a functional fragment thereof. More preferably, it is a human antibody comprising a heavy chain variable region (V_{H}) having the amino acid sequence SEQ ID NO: 21 and/or a light chain variable region (V_{L}) having the amino acid sequence SEQ ID NO:22.

In another aspect, the present invention therefore relates to the antibody anti-hFcγRIIA IV.3, a chimeric form thereof, a humanized form thereof, a human form thereof, or a functional fragment thereof, for use for preventing and/or treating IgG antibody-dependent inflammatory and autoimmune disorders.

Preferably, these inflammatory and autoimmune disorders are chosen in the group consisting of: arthritic symptoms, allergic reactions, lupus or antibody-nephritis.

In a preferred embodiment, said inflammatory disorder is rheumatoid arthritis.

In another preferred embodiment, said inflammatory disorder is anaphylaxis.

The present invention also relates to the use of the antibody anti-hFcγRIIA IV.3, a chimeric form thereof, a humanized form thereof, a human form thereof, or a functional fragment thereof, for the manufacture of a pharmaceutical composition intended to prevent and/or treat IgG antibody-dependent inflammatory and autoimmune disorders, in subjects in need thereof.

In other words, the present invention relates to a method for treating a subject suffering from an IgG antibody-dependent inflammatory and autoimmune disorder, comprising the administration of an efficient amount of the antibody anti-hFcγRIIA IV.3, a chimeric form thereof, a humanized form thereof, a human form thereof, or a functional fragment thereof.

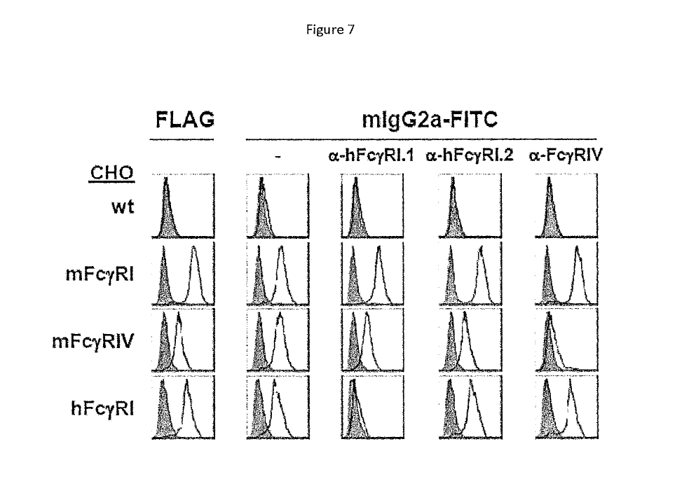

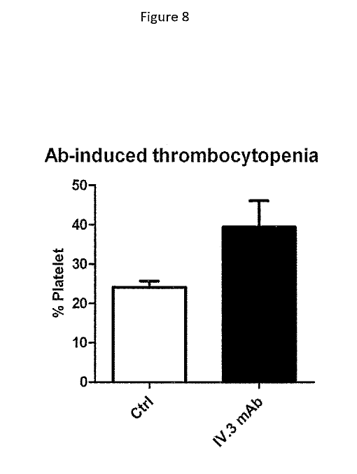

The present inventors have also demonstrated that the blocking antibody anti-hFcγRIIA IV.3 is also able to treat thrombocytopenia in animal model (see FIG. 8).

In another aspect, the present invention therefore also relates to the antibody anti-hFcγRIIA IV.3, a chimeric form thereof, a humanized form thereof, a human form thereof, or a functional fragment thereof, for use for preventing and/or treating thrombocytopenia.

The present invention also relates to the use of the antibody anti-hFcγRIIA IV.3, a chimeric form thereof, a humanized form thereof, a human form thereof, or a functional fragment thereof, for the manufacture of a pharmaceutical composition intended to prevent and/or treat thrombocytopenia, in subjects in need thereof.

In other words, the present invention relates to a method for treating a subject suffering from thrombocytopenia, comprising the administration of an efficient amount of the antibody anti-hFcγRIIA IV.3, a chimeric form thereof, a humanized form thereof, a human form thereof, or a functional fragment thereof.

Preferably, the said antibody or fragment will be administered by systemic route, notably by intravenous, intramuscular, intradermal, intraperitoneal, subcutaneous or oral route. More preferably, the composition composed of the antibody of the invention will be administered in several doses spaced equally over time. Their administration routes, dosing schedules and optimal galenic forms can be determined according to the criteria generally taken into account when establishing a treatment suited to a patient such as, for example, the patient's age or body weight, the seriousness of his general state, his tolerance for the treatment and the side effects experienced.

Preferably, said subject in a human.

Combination Product of the Invention

As disclosed previously, the present inventors demonstrated that two blocking monoclonal antibodies, namely the monoclonal anti-hFcγRI.1 antibody and the monoclonal anti-hFcγRIIA IV.3 antibody, prevent and even abolish airway inflammation, systemic anaphylaxis, autoimmune arthritis and thrombocytopenia in animal models.

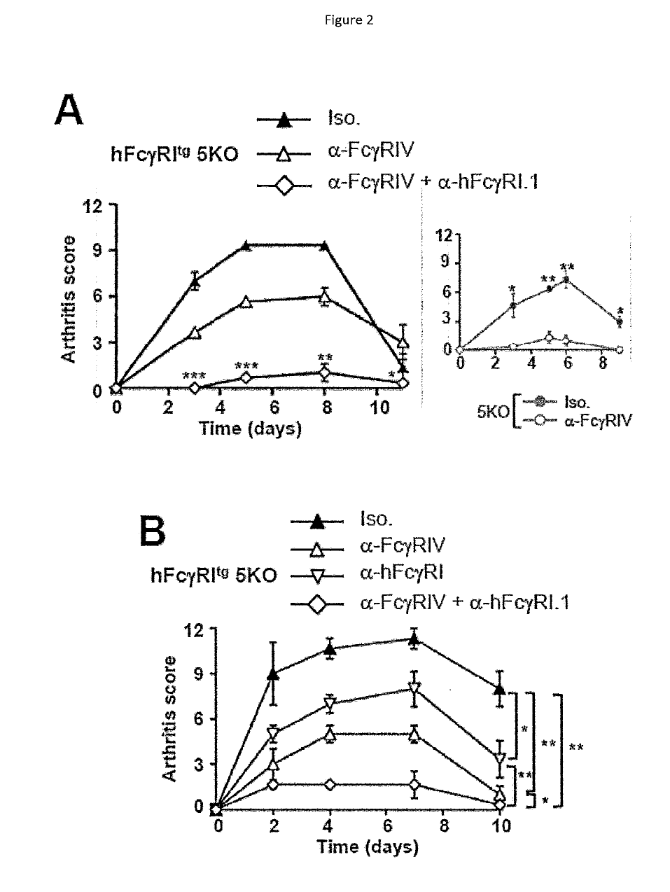

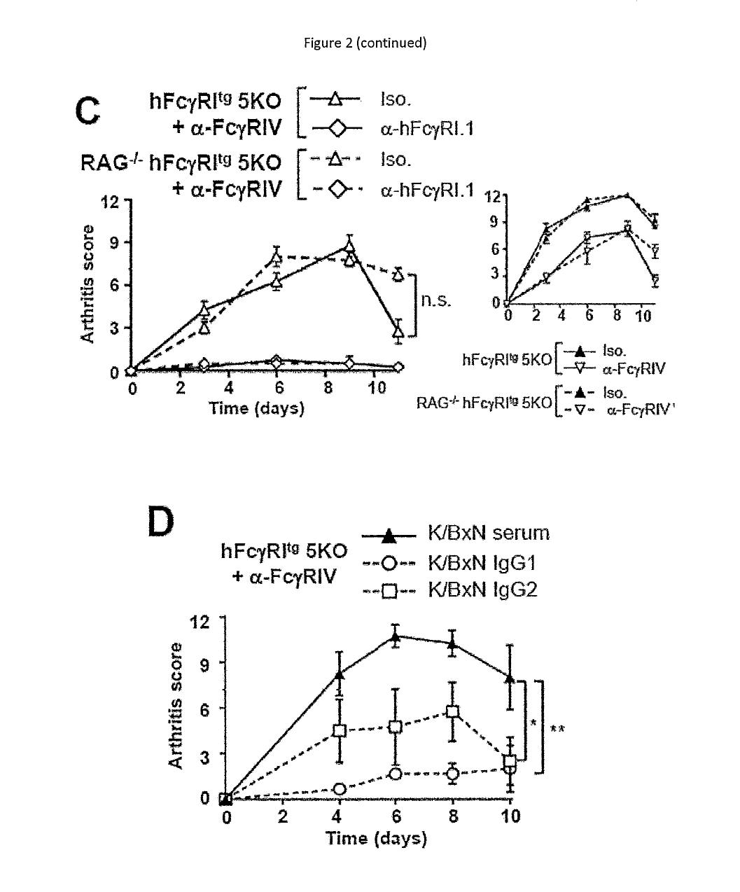

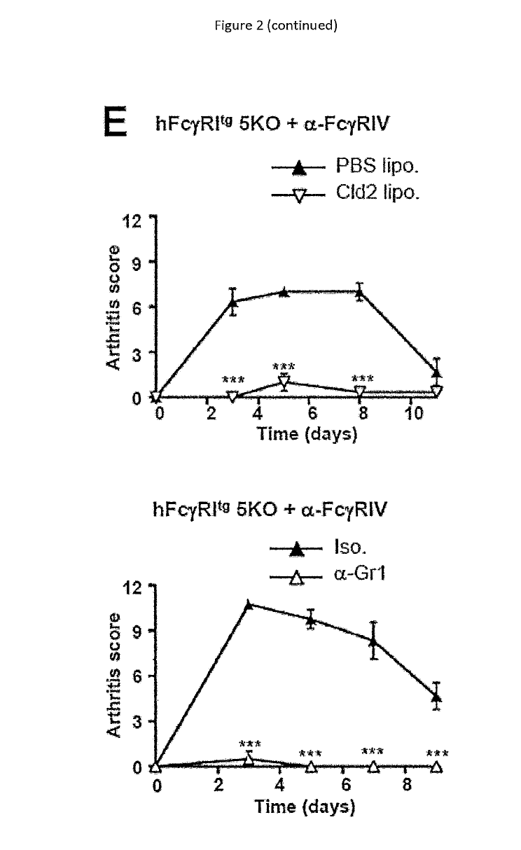

Furthermore, it has been observed that the blocking of two IgG receptors (demonstrated for hFcγRI and FcγRIV, see FIGS. 2A, 3A, 4D, 5B, 6B) has an additive effect to reduce the symptoms of these diseases. The same principle should apply to hFcγRIIA and hFcγRI. The inventors thus propose to block these two human receptors concomitantly for efficiently preventing and/or treating these diseases. In this aim, it will be advantageous to use the antibodies described above, since they were shown to efficiently block the two receptors hFcγRIIA and hFcγRI.

Hence, the inventors propose to use both the blocking anti-hFcγRI.1 antibody of the invention and the blocking anti-hFcγRIIA IV.3 antibody, chimeric forms thereof, humanized forms thereof, human forms thereof or functional fragments thereof, in a pharmaceutical combination product that is intended to prevent and/or treat IgG antibody-dependent inflammatory and autoimmune disorders or thrombocytopenia.

In another aspect, the present invention therefore relates to a therapeutic substance combination product containing the antibody of the invention, or a functional fragment thereof, and a compound blocking the human FcγRIIA receptor, preferably of SEQ ID NO:8 and SEQ ID NO:27.

Said compound can be any chemical or biological compound that is known to i) specifically bind the human FcγRIIA receptor, and ii) block this receptor efficiently (that is, either by inhibiting the binding of said receptor with all his natural ligand(s), or by impairing the signaling pathway generated by said binding and the subsequent activation of the cell carrying the said receptor).

In a preferred embodiment, said compound is a monoclonal antibody or a fragment thereof.

In a more preferred embodiment, said monoclonal antibody comprises at least one, preferably two, preferably three, preferably four, preferably five and even more preferably six CDR(s) of the antibody IV.3, that are:

(SEQ ID NO: 11)
the heavy chain CDR1: GYTFTNYG;
(SEQ ID NO: 12)
the heavy chain CDR2: LNTYTGES;
(SEQ ID NO: 13)
the heavy chain CDR3: ARGDYGYDDPLDY,
(SEQ ID NO: 14)
the light chain CDR1: KSLLHTNGNTY
(SEQ ID NO: 15)
the light chain CDR2: RMSV;
and
(SEQ ID NO: 16)
the light chain CDR3: MQHLEYPLT.

In a more preferred embodiment, said monoclonal antibody comprises a heavy chain variable region (V_{H}) having the amino acid sequence SEQ ID NO: 17 and/or a light chain variable region (V_{L}) having the amino acid sequence SEQ ID NO: 18.

In another preferred embodiment, said compound is a chimeric form of said antibody, a humanized form of said antibody, a human form of said antibody, or a functional fragment thereof.

In another aspect, the present invention relates to the therapeutic substance combination product of the invention, for simultaneous, separate or sequential use, as a medicament for preventing and/or treating IgG antibody-dependent inflammatory and autoimmune disorders such as arthritic symptoms, allergic reactions, lupus or antibody-nephritis, preferably rheumatoid arthritis and anaphylaxis.

The present invention also relates to a method for preventing and/or treating IgG antibody-dependent inflammatory and autoimmune disorders such as arthritic symptoms, allergic reactions, lupus or antibody-nephritis, preferably rheumatoid arthritis and anaphylaxis, comprising the administration, in a subject in need thereof, of an efficient amount of the antibody of the invention or of a functional fragment thereof, and an efficient amount of a compound blocking hFcγRIIA. This compound is preferably a humanized antibody comprising at least one, preferably two, preferably three, preferably four, preferably five and even more preferably six CDR(s) of the antibody IV.3 (SEQ ID NO:11 to 16). More preferably, this compound is a humanized antibody comprising a heavy chain variable region (V_{H}) having the amino acid sequence SEQ ID NO: 21 and/or a light chain variable region (V_{L}) having the amino acid sequence SEQ ID NO: 22. This administration can be concomitant or sequential.

In another aspect, the present invention relates to the therapeutic substance combination product of the invention, for simultaneous, separate or sequential use, as a medicament for preventing and/or treating thrombocytopenia.

The present invention also relates to a method for preventing and/or treating thrombocytopenia, comprising the administration, in a subject in need thereof, of an efficient amount of the antibody of the invention or of a functional fragment thereof, and an efficient amount of a compound blocking hFcγRIIA. This compound is preferably a humanized antibody comprising at least one, preferably two, preferably three, preferably four, preferably five and even more preferably six CDR(s) of the antibody IV.3 (SEQ ID NO:11 to 16). More preferably, this compound is a humanized antibody comprising a heavy chain variable region (V_{H}) having the amino acid sequence SEQ ID NO: 21 and/or a light chain variable region (V_{L}) having the amino acid sequence SEQ ID NO: 22. This administration can be concomitant or sequential.

In the case of simultaneous use, the two components of the treatment (the antibody of the invention and the compound blocking hFcγRIIA) are administered to the patient simultaneously. According to this embodiment, the two components can be packaged together, i.e., in the form of a mixture. The two components can also be packaged separately, then optionally mixed before being administered together to the patient. More commonly, the two components are administered separately or sequentially. They can for example be administered separately or sequentially with an interval of time which is typically comprised between few minutes and several hours, preferably between 1 minute and five hours, more preferably between 1 minute and two hours. As the half-life of the antibodies of the invention is of 21 days in vivo, it is also possible to administer the two components of the therapeutic combination product of the invention with an interval of time of one to several days, typically of one to ten days.

In a particular embodiment, the therapeutic substance combination product of the invention is a single pharmaceutical composition containing, in the same recipient, the two active principles (the antibody of the invention and the compound blocking hFcγRIIA). Alternatively, the two active principles of the combination product can be separated in two different recipients and administered concomitantly (they are mixed extemporaneously) or separately. In particular, the routes of administration of the two components may be different. The administration can also be performed at different sites.

In another aspect, the present invention therefore discloses a pharmaceutical composition containing an efficient amount of the antibody of the invention, as defined above, or of a functional fragment thereof, and an efficient amount of a compound blocking the human FcγRIIA receptor of SEQ ID NO:8 and SEQ ID NO:27. Said compound is preferably a humanized antibody comprising at least one, preferably two, preferably three, preferably four, preferably five and even more preferably six CDR(s) of the antibody IV.3 (SEQ ID NO:11 to 16). More preferably, this compound is a humanized antibody comprising a heavy chain variable region (V_{H}) having the amino acid sequence SEQ ID NO: 21 and/or a light chain variable region (V_{L}) having the amino acid sequence SEQ ID NO: 22. The said pharmaceutical composition may also contain any pharmaceutically acceptable carrier or excipient.

### Examples

### I. Role of Anti-hFcγRI.1. In the Treatment of Inflammatory-Related Disorders

To investigate the role of human FcγRI in vivo, transgenic mice for this receptor were used, that display an expression pattern of hFcγRI comparable to that found in humans. To avoid a possible in vivo competition or contribution of endogenous FcγRs to reactions mediated by hFcγRI, hFcγRI-transgenic mice were crossed with 5KO mice that lack FcγRI, FcγRIIB, FcγRIII, FcεRI and FcεRII. The resulting hFcγRI^{tg} 5KO mice express only two activating FcRs, transgenic hFcγRI and endogenous FcγRIV that could be efficiently blocked in vivo to study the specific contribution of hFcγRI to a particular disease or therapy model. The expression of the transgene in this background lead to an increased expression level of hFcγRI on neutrophils in transgenic mice compared to humans, but a very similar expression on monocytes. Monocytes, macrophages and dendritic cells in humans and in these transgenic mice indeed express hFcγRI. Noticeably, however, hFcγRI was reported to be inducible on human neutrophils whereas neutrophils from hFcγRI^{tg} mice constitutively express hFcγRI. Nevertheless, hFcγRI was reported to be expressed on human neutrophils under multiple circumstances including, in particular rheumatoid arthritis and multiple myeloma. One can therefore consider that human neutrophils may express hFcγRI in most inflammatory contexts.

hFcγRI bound not only human IgG1/3/4 subclasses but also mouse IgG2a/2b subclasses as monomers. Importantly, the affinity of hFcγRI for mIgG2a was very similar to its affinity for hIgG1 (K_{D}≈38 nM and 40 nM, respectively), in the range of the high-affinity mIgG2a-mFcγRIV interaction (K_{D}≈34 nM). hFcγRI thus functions as a high-affinity IgG receptor not only in humans but also in hFcγRI^{tg} mice. The fact that hFcγRI conserved its high-affinity properties also for mouse IgG validates hFcγRI^{tg} mice as a model to study the contribution of hFcγRI to disease and therapy.

I.1. Material and Methods

Mice

FcγRI/IIIB/IIIA^{-/-} FcεRI^{-/-} FcεRII^{-/-} (5KO) mice have been described (Mancardi D A, J. Clin. Invest. 2008, 118(11):3738-3750). hFcγRI^{tg} mice were obtained from J. G. J. van de Winkel (UMCU, Utrecht, The Netherlands) and crossed to 5KO mice to obtain hFcγRI^{tg} 5 KO. These mice were further crossed to RAG^{-/-} mice to generate RAG^{-/-} hFcγRI transgenic 5KO mice. All mice carrying the hFcγRI transgene were used as heterozygous animals and non-transgenic littermates served as controls. KRN^{tg} mice were provided by D. Mathis, C. Benoist (HMS, Boston, Mass., USA), and IGBMC (Strasbourg, France). Mice used in experiments were on C57BL/6J background (6th-12th generation backcross). WT mice were purchased from Charles River. All mouse protocols were approved by the Animal Care and Use Committees of Paris, Ile de France, France.

Reagents

Anti-mouse CD11b, CD11c, CD3, CD19, Gr1, SiglecF, CD117, DX5, CD61, NK1.1, IgE and labeled anti-hFcγRI were from BD Biosciences; mouse IgG3 anti-DNP from Serotec; HRP-coupled anti-mouse IgG subclasses from Southern Biotechnology; anti-FLAG mAbs, OVA, BSA, rabbit GPI, rabbit anti-ova antiserum, gadolinium-(III)-chloride, Freund's adjuvant, ABT-491, cyproheptadine from Sigma-Aldrich, MPO ELISA kit from HyCult Biotech. IgG were purified by Protein G-affinity purification from supernatants of hybridomas producing anti-hFcγRI.1 mAb, anti-mFcγRIV mAb provided by J. V. Ravetch (Rockefeller University, New York, N.Y., USA), anti-Gr1 mAb provided by R. Coffman (DNAX, Palo Alto, Calif., USA), anti-DNP mIgG1, IgG2a and mIgG2b provided by B. Heyman (Uppsala Universitet, Uppsala, Sweden) and anti-platelet mAb 6A6 provided by Dr R. Good (USFCM, Tampa, Fla., USA). Purified anti-hFcγRI.2 mAb (clone 10.1) was provided by N. Hogg (CRUK, London, UK). PBS-liposomes and Clodronate-liposomes were prepared as published (Van Rooijen N. et al, J. Immunol. Methods 1994; 174(1-2):83-93). CHO K1 cells stably transfected with FLAG-tagged mouse FcγRs or human FLAG-tagged FcγRs were cultured as described in Mancardi et al, J. Clin. Invest. 2008 (118(11):3738-3750) and Bruhns P, Blood 2009 (113:3716-3725).

Anti-GPI IgG were purified from K/B×N serum using Protein G, polyclonal IgG1 and IgG2 fractions using anti-mIgG1 or anti-mIgG2 sepharose beads (Nordic Immunology). IgG subclasses were determined by ELISA; IgG1, IgG2a and IgG2b anti-GPI mAbs obtained in collaboration with the Antibody Production Platform (Institut Pasteur, Paris, France) were used as standards.

Flow Cytometry Analysis

Blood cells populations were defined as follows: B cells (CD19⁺), T cells (CD3⁺), monocytes/macrophages (blood/peritoneum: CDIIIB⁺/Gr1⁻; BAL: CD11c⁺/Gr1⁻), neutrophils (Gr1⁺/SiglecF⁻), basophils (IgE⁺/DX5⁺), eosinophils (Gr1i^{nt}/SiglecF⁺), mast cells (IgE⁺/CD117⁺), platelets (DX5⁺/CD61⁺), NK cells (NK1.1⁺/DX5⁺), Human B cells (CD19+), T cells (CD3+), NK cells (CD56+), monocytes (CD14+) neutrophils (CD24+), basophils (CD123+/CD203c+), and eosinophils (CD24+/CD193+). Expression of different Flag-tagged FcRs in CHO-K1 cells was compared using anti-FLAG antibody.

Immune complex binding: CHO-K1 cells were incubated with preformed ICs made of 10 μg/ml TNP₅—BSA-biotin and 15 μg/ml anti-TNP monoclonal antibodies, for 1 hour at 4° C. Bound ICs were detected using PE-conjugated neutravidin at 2 μg/ml, for 30 minutes at 4° C.

Monomeric Ig Binding Assays:

CHO-K1 cells were incubated with 10 μg/ml monomeric mouse IgG or rabbit IgG for 1 hour at 4° C. Cell-bound Ig was detected using 5 μg/ml PE-labeled F(ab′)₂ fragments of anti-mouse F(ab′)₂-specific or 15 μg/ml FITC-conjugated F(ab′)₂ anti-rabbit Ig, respectively, for 30 minutes at 4° C.

K/B×N Serum-Induced Passive Arthritis (K/B×N PA)

K/B×N serum was generated. Arthritis was induced by an intravenous injection of 150 μL of K/B×N serum and arthritis was scored as described (Bruhns P. et al, Immunity 2003; 18(4):573-581).

In Vivo Blocking and Depletion

200 μg/mouse of anti-FcγRIV or anti-hFcγRI.1 blocking mAbs were injected i.v. once 30 min before the beginning of the experiment, except for arthritis were blocking antibodies were injected every second day.

500 μg/mouse of anti-Gr1 mAbs, 300 μl/mouse of PBS- or clodronate-liposomes (at 2.1 mg/mouse), 1 mg/mouse of GdCl3 were injected i.v. 24 hours before the beginning of the experiment, except for arthritis were anti-Gr1 mAbs and liposomes were injected every second day.

ABT-49 (25 μg/mouse) or cyproheptadine (50 μg/mouse) was injected i.v. 20 or i.p. 30 min before challenge, respectively. Depletion of specific populations was ascertained using flow cytometry on blood samples taken during or after the experiment (data not shown).

Airway Inflammation

Mice were injected intranasally with 20 μl of rabbit anti-OVA antiserum and i.v. with 500 μg OVA. After 18 hours, mice were lethally anesthetized and four broncho-alveolar lavages of respectively 0.5, 1, 1 and 1 ml PBS were performed. The supernatant of the first lavage was used to quantify MPO content. The cells from all lavages were pooled for cell count analysis. Hemorrhage was determined in the cell-free supernatant of pooled lavages after RBC lysis by optical density measurement (570 nm).

Anaphylaxis

PSA:

Immune complexes made of 80 μg GPI and 200 μl anti-GPI containing serum (K/B×N serum) in 300 μl physiological solution were pre-formed at 37° C. and injected i.v. Alternatively, 10 to 200 μg of anti-hFcγRI.1 or anti-hFcγRI.2 mAbs was injected i.v. Central body temperature was recorded using a digital thermometer (YSI).

ASA:

Mice were injected i.p. on day 0 with 200 μg BSA in CFA and boosted i.p. on day 14 and day 28 with 200 μg BSA in IFA. BSA-specific IgG1, IgG2a/b/c and IgE antibodies in serum were titered by ELISA on day 30 as described (Jonsson F. et al, J. Clin. Invest. 2011; 121(4):1484-1496). Mice with comparable antibody titers were challenged i.v. with 500 μg BSA, 8 days after the last immunization. Central temperature was monitored.

Experimental Thrombocytopenia (ITP)

Blood samples were taken retro-orbitally before, and at indicated time points after the i.v. injection of 5 μg of anti-platelet mAb. Platelet counts were determined using an ABC Vet automatic blood analyzer (Horiba ABX).

Statistical Analyses

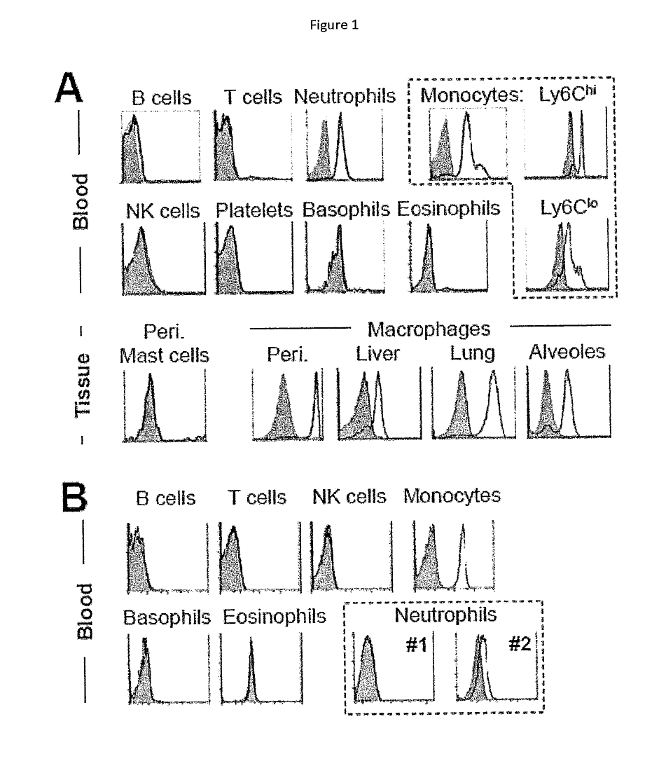

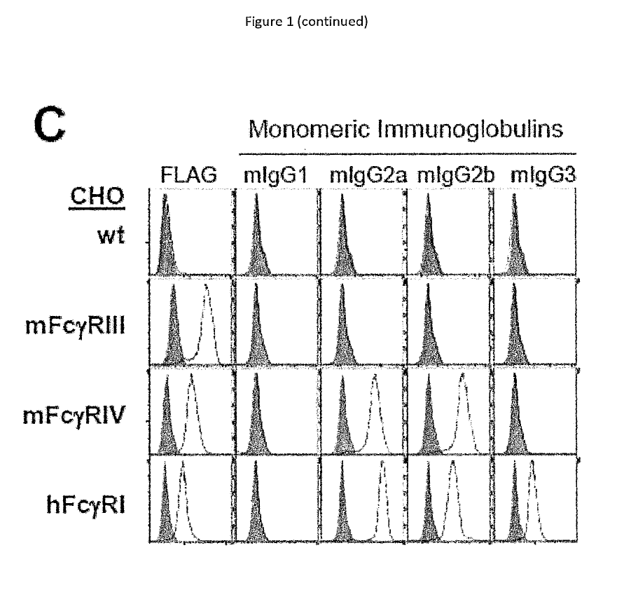

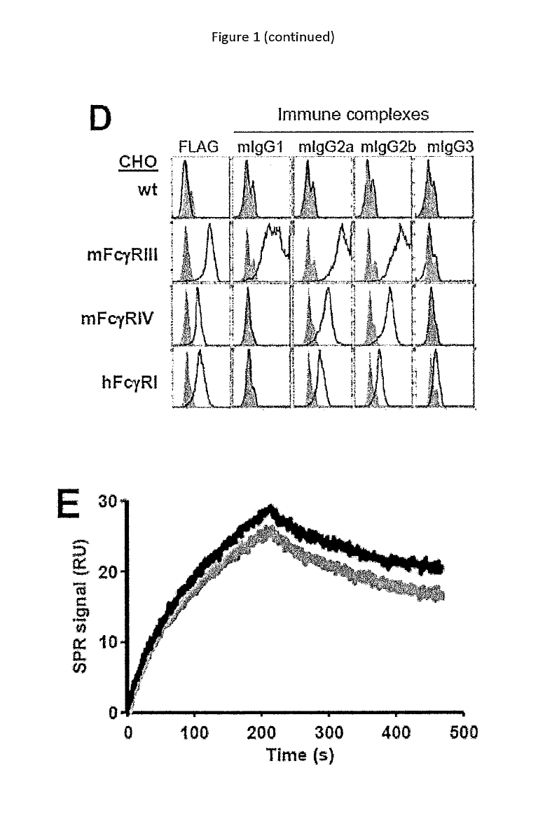

Data was analyzed using one-way ANOVA with Bonferroni post-test (FIGS. 1E-F, 2, 3A-C, 5A, 6), two-way ANOVA with Bonferroni post-test (FIG. 1G, 5B-E), Mantel Cox test for all Survival curves or Student's t-test (all other data). Statistical significance is indicated (ns: p>0.05; *: p<0.05; **: p<0.01; ***: p<0.001). The n given in the Figure Legends corresponds to the number of mice per group in individual experiments.

I.2. Results

hFcγRI was found sufficient to trigger autoimmune arthritis and thrombocytopenia, immune complex-induced airway inflammation, active and passive systemic anaphylaxis. Monocyte/macrophages were identified to be responsible for thrombocytopenia, neutrophils to be responsible for systemic anaphylaxis, and both cell types to be responsible for arthritis induction.

These results are detailed below.

Efficient Blockade of the Human FcγRI Receptor by the Antibody of the Invention

The anti-hFcγRI.1 monoclonal antibody is a mouse IgG2a having V_{H} and V_{L} sequences described in SEQ ID NOs: 9 and 10 respectively. Its affinity constant (Kₐ) for the hFcγRI receptor is of 1.5×10⁹M⁻¹.

The anti-hFcγRI.2 monoclonal antibody is the clone 10.1 sold by ebioscience, Milipore, and Invitrogen.

To investigate the ability of anti-hFcγRI monoclonal antibodies to block ligand binding (i.e., IgG binding) to hFcγRI, we used an in vitro binding assay. We reported earlier collections of CHO-K1 cells transfected with a mouse or a human FcγR (Mancardi et al, J. Clin. Invest. 2008 (118(11):3738-3750) and Bruhns P, Blood 2009 (113:3716-3725)). The binding of FITC-conjugated IgG2 to mouse FcγRI, mouse FcγRIV (used as controls) or to human FcγRI-expressing CHO transfectants was investigated following pre-incubation or not with anti-hFcγRI.1 mAb, anti-hFcγRI.2 mAb or anti-FcγRIV mAb. All three transfectants bound FITC-conjugated IgG2 (FIG. 7).

Anti-hFcγRI.1 mAb, but not anti-hFcγRI.2 mAb, abolished FITC-conjugated IgG2 binding specifically to hFcγRI. Anti-hFcγRI.2 demonstrated not blocking ability, whereas anti-mFcγRIV mAb (clone 9E9) efficiently blocked FITC-conjugated IgG2 binding to mFcγRIV (FIG. 7).

hFcγRI.1 mAb is therefore able to block 100% of IgG-immune complex binding to hFcγRI. It is therefore a specific blocking mAb against hFcγRI.

N.B. In all further experiments, in vivo hFcγRI blockade will be achieved by anti-hFcγRI.1. mAb injections (see FIG. 7)

hFcγRI can Trigger Passive Inflammatory Arthritis.

To investigate the pro-inflammatory potential of hFcγRI in vivo mice transgenic for hFcγRI (hFcγRI^{tg}) were crossed to mice deficient for five endogenous FcRs (FcγRI/IIIB/III^{-/-} FcεRI/II^{-/-} mice, aka 5KO mice). These mice still express the FcRγ-chain that is mandatory for hFcγRI expression and endogenous FcγRIV. In hFcγRI^{tg} 5KO mice, hFcγRI was expressed in the blood specifically on Ly6C^{hi} and Ly6C^{lo} monocytes, on neutrophils, and on peritoneal, liver, lung and alveolar macrophages, but not on peritoneal mast cells (FIG. 1A), in agreement with a previous report (Heijnen I A. et al, J. Clin Invest. 1996). The expression pattern of hFcγRI in hFcγRI^{tg} 5KO mice therefore mimics its expression pattern in humans in which hFcγRI is constitutively expressed on monocytes and inducible on neutrophils. Noticeably, whereas the expression level of hFcγRI was higher on neutrophils from these mice compared to human neutrophils from two different normal donors, it was similar on mouse monocytes compared to monocytes from normal donors (FIG. 1B). Importantly, hFcγRI bound mouse IgG2a, IgG2b and IgG3, but not mouse IgG1, either as monomers (FIG. 1C) or as immune complexes (FIG. 1D). Moreover, the analysis of the interaction of hFcγRI with mouse IgG2a or with human IgG1 resulted in similar association (kₒₙ) and dissociation (k_{off}) constants, and therefore in a very similar calculated affinity constant (K_{D}≈40 nM, i.e. K_{A}≈2.5×10⁷M⁻¹) (FIG. 1E). The kinetic parameters determined from experiments presented in FIG. 1E are:

| | kon | koff | Half life | K_{D} |
|---|---|---|---|---|
| F | (10⁴ M⁻¹s⁻¹) | (10⁻³ s⁻¹) | (s) | (nM) |
| hIgG1 | 3.5 ± 0.8 | 1.4 ± 0.2 | 216 ± 17 | 41 ± 14 |
| mIgG2a | 3.6 ± 0.6 | 1.1 ± 0.1 | 271 ± 30 | 38 ± 8 |

hFcγRI therefore retains its properties as a high-affinity receptor for IgG (i.e. for human IgG1, IgG3 and IgG4) when expressed in transgenic mice (i.e. high-affinity for mouse IgG2a, IgG2b and IgG3).

It was then investigated whether hFcvRI could induce arthritic inflammation using hFcγRI^{tg} 5KO mice and K/B×N serum that contains pathogenic IgG2 anti-GPI antibodies. The serum of spontaneously arthritic K/B×N mice (F1 offsprings from KRN^{tg} mice crossed with NOD mice) indeed contains pathogenic IgG1 and IgG2 anti-Glucose-6-Phosphate Isomerase (GPI) antibodies able to form immune complexes with GPI deposited on the articular cartilage. These immune complexes induce inflammatory arthritis that requires activating FcγRs. Both 5KO and hFcγRI^{tg} 5KO mice developed arthritis (FIG. 2A) following K/B×N serum injection (K/B×N PA). Blocking FcγRIV using blocking anti-FcγRIV mAbs abolished arthritis in 5KO, but not in hFcγRI^{tg} 5 KO mice. Blocking FcγRIV using anti-FcγRIV mAbs and hFcγRI using blocking anti-hFcγRI.1 mAbs was necessary to abolish K/B×N PA in hFcγRI^{tg} 5KO mice (FIG. 2A). Blocking hFcγRI significantly reduced arthritis symptoms in hFcγRI^{tg} 5KO mice (FIG. 2B).

hFcγRI-dependent arthritis (arthritis developing in anti-FcγRIV-treated hFcγRI^{tg} 5KO mice) was milder than arthritis developing in untreated hFcγRI^{tg} 5KO mice. Occupancy of a proportion of this human high-affinity receptor by endogenous mouse IgG may be responsible for these mild arthritic symptoms. hFcγRI-dependent arthritis did not, however, increase in severity when induced in RAG-deficient hFcγRI^{tg} 5KO mice that lack endogenous IgG (FIG. 2C). Similar results were obtained for FcγRIV-dependent arthritis (FIG. 1F, insert). If occurring in vivo, partial occupancy or saturation of hFcγRI (or FcγRIV) by IgG does therefore not affect K/B×N arthritis induction and development. As expected, IgG2 antibodies purified from K/B×N serum induced hFcγRI-dependent arthritis, whereas IgG1 antibodies purified from K/B×N serum induced only very modest pathological symptoms (FIG. 2D). Finally, hFcγRI-dependent arthritis was abolished when monocytes/macrophages or neutrophils were depleted (FIG. 2E). Altogether, these results demonstrate that hFcγRI is sufficient to induce K/B×N passive arthritis, mediated by mouse IgG2 autoantibodies, that required both monocytes/macrophages and neutrophils.

hFcγRI can Trigger Antibody-Dependent Airway Inflammation

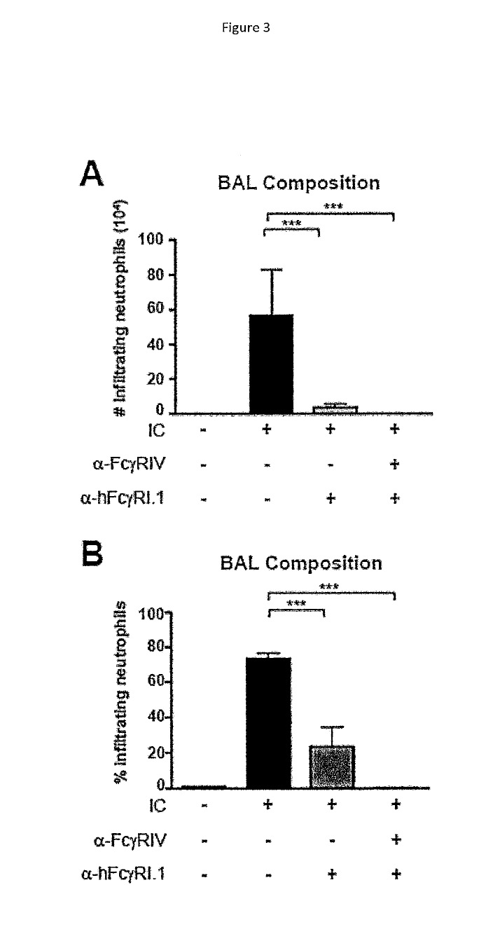

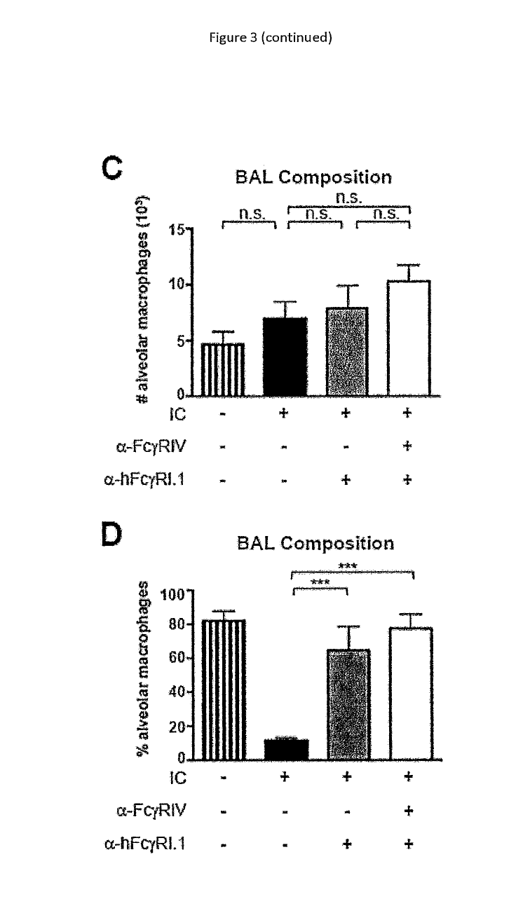

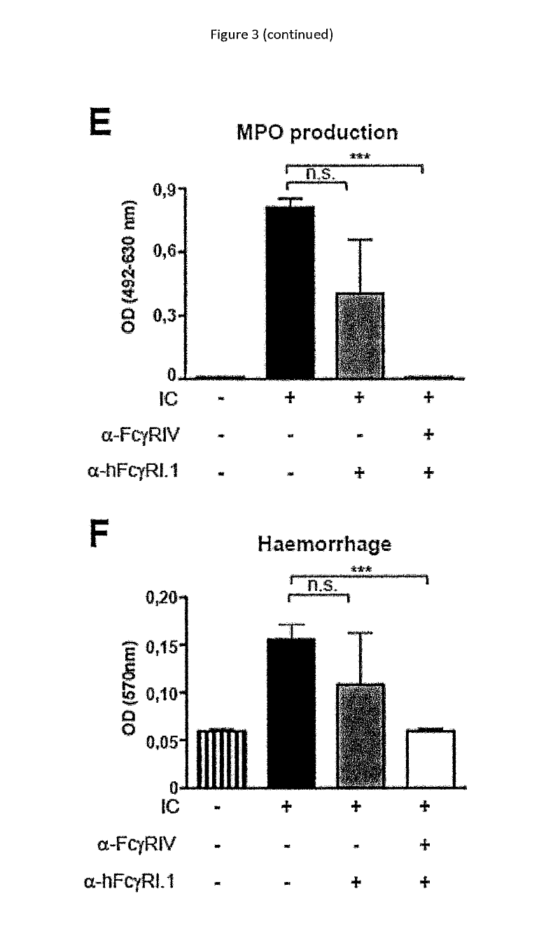

As hFcγRI is expressed on lung and alveolar macrophages from hFcγR^{tg} 5KO mice (FIG. 1A), it was next investigated if hFcγRI could induce lung inflammation in a model of immune complex-mediated airway inflammation. This disease model of a reverse Arthus reaction consists of an intravenous injection of antigen (OVA) and of intranasal instillation of anti-OVA antibodies that was shown to depend on the expression of activating FcRs on alveolar macrophages (Skokowa J, et al. J Immunol. 2005). Intravenous injection of OVA followed by intranasal instillation of rabbit anti-OVA serum (hFcγRI binds rabbit IgG, data not shown) lead to a massive infiltration of neutrophils in the airways within 18 hours, as determined in broncho-alveolar lavages (BAL). Whereas blocking either hFcγRI or mFcγRIV significantly inhibited neutrophil infiltration, blocking both hFcγRI and FcγRIV was necessary to abolish neutrophil infiltration (FIG. 3A,B). No significant variation in alveolar macrophage numbers under these different conditions was observed (FIG. 3C). When occurring however, neutrophil infiltration drastically modified the alveolar macrophage/neutrophil ratio in BAL (FIG. 3D vs 3B). Similarly myeloperoxidase production in the BAL (FIG. 3E), resulting from neutrophil and/or macrophage activation, and hemorrhage (FIG. 3F), resulting from tissue damage, had a trend to be reduced following hFcγRI blockade and was significantly reduced following mFcγRIV blockade, both symptoms were abolished following blockage of both receptors. Altogether, these results demonstrate that hFcγRI is sufficient to induce airway inflammation.

hFcγRI can Trigger Passive Systemic Anaphylaxis.

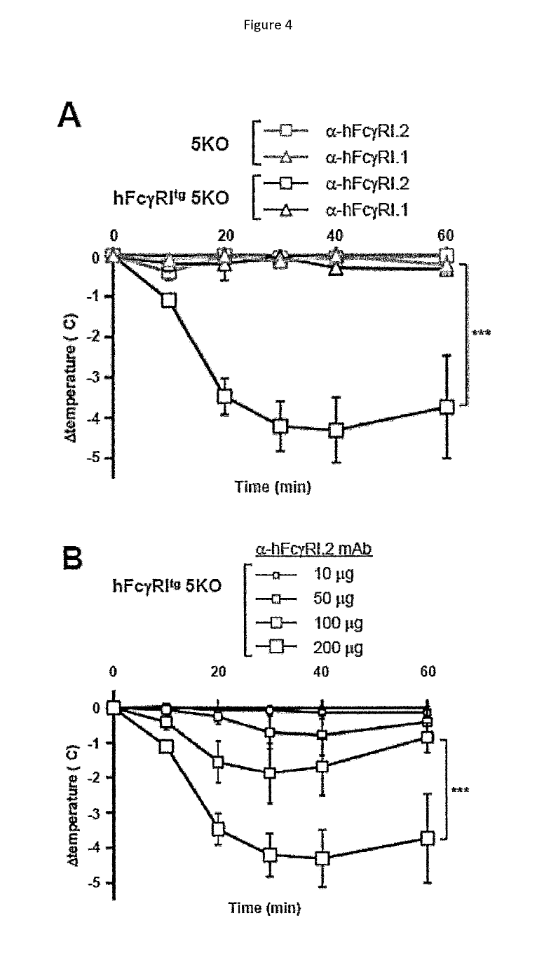

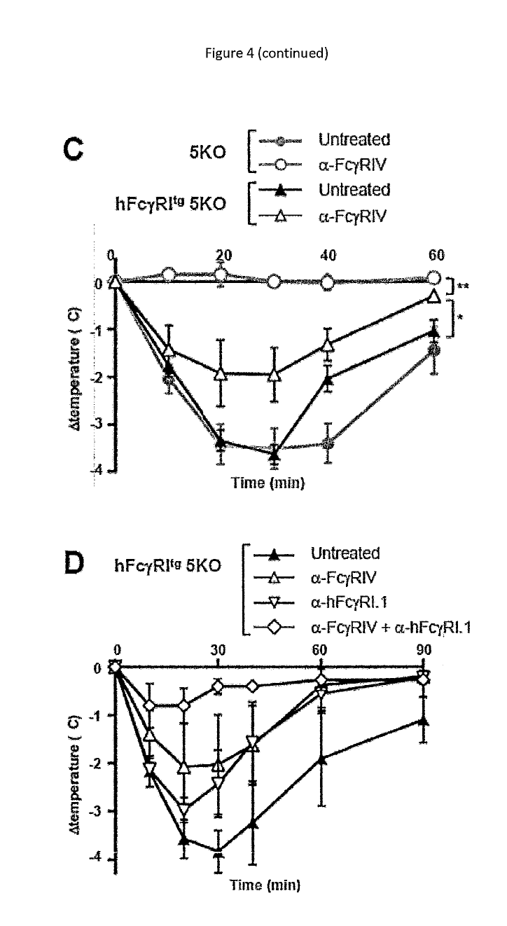

It was recently reported that FcγRIV was responsible for IgG2b-induced passive systemic anaphylaxis (PSA) that arises following intravenous injection of preformed immune complexes made of mouse IgG2b (anti-DNP) and antigen (DNP-BSA). The potential of hFcγRI, which has the same expression pattern and ligands as FcγRIV in transgenic mice to induce PSA in hFcγRI^{tg} 5KO mice using divalent (anti-hFcγRI mAbs) or multivalent (IgG-immune complexes) ligands, was therefore investigated. An i.v. injection of the non-blocking anti-hFcγRI.2 mAb, but not of the blocking anti-hFcγRI.1 mAb, induced a significant temperature drop in hFcγRI^{tg} 5KO mice, but not in 5KO mice (FIG. 4A). The effect of the non-blocking anti-hFcγRI.2 mAb injection on the central temperature of hFcγR^{tg} 5KO mice was dose-dependent (FIG. 4B) and resulted in fatal anaphylactic shocks at higher doses (data not shown). Therefore, whereas anti-hFcγRI.1 mAb is an antagonistic blocking antibody, anti-hFcγRI.2 mAb is an agonistic non-blocking antibody capable of inducing hFcγRI-dependent anaphylaxis.

An i.v. injection of mouse IgG2b-immune complexes induced a temperature drop in 5KO and hFcγRI^{tg} 5KO mice that was abolished by FcγRIV blockade in 5KO, but not in hFcγRI^{tg} 5KO mice (FIG. 4C). Confirming the anaphylactogenic potential of hFcγRI, blocking hFcγRI reduced the temperature drop in hFcγRI^{tg} 5KO mice. hFcγRI-dependent PSA (anaphylaxis developing in anti-FcγRIV-treated hFcγRI^{tg} 5KO mice) was abrogated by hFcγRI blockade (FIG. 4D). Altogether, these results demonstrate that hFcγRI is sufficient to trigger PSA in transgenic mice.

Neutrophils and PAF Mediate hFcγRI-Dependent Active Systemic Anaphylaxis.

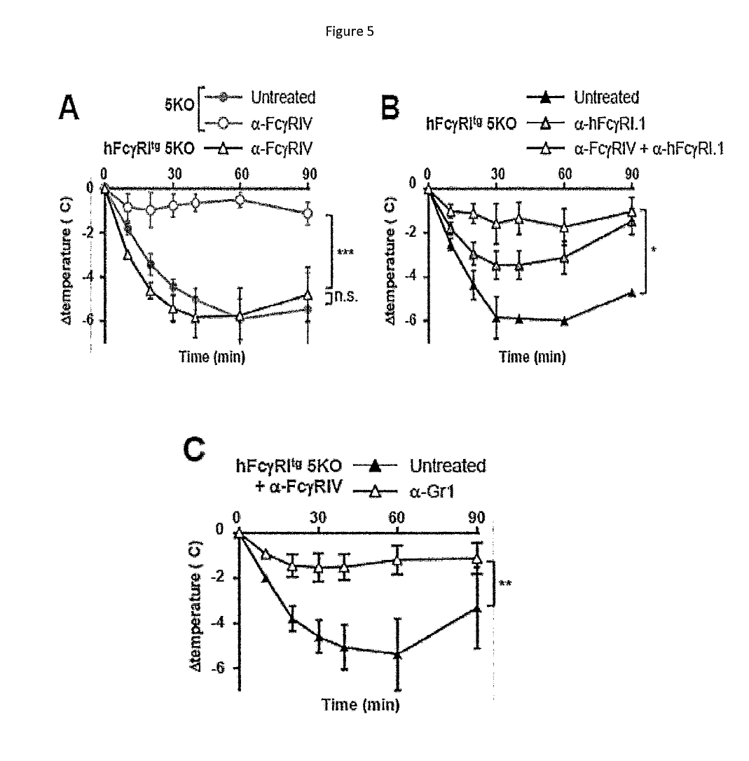

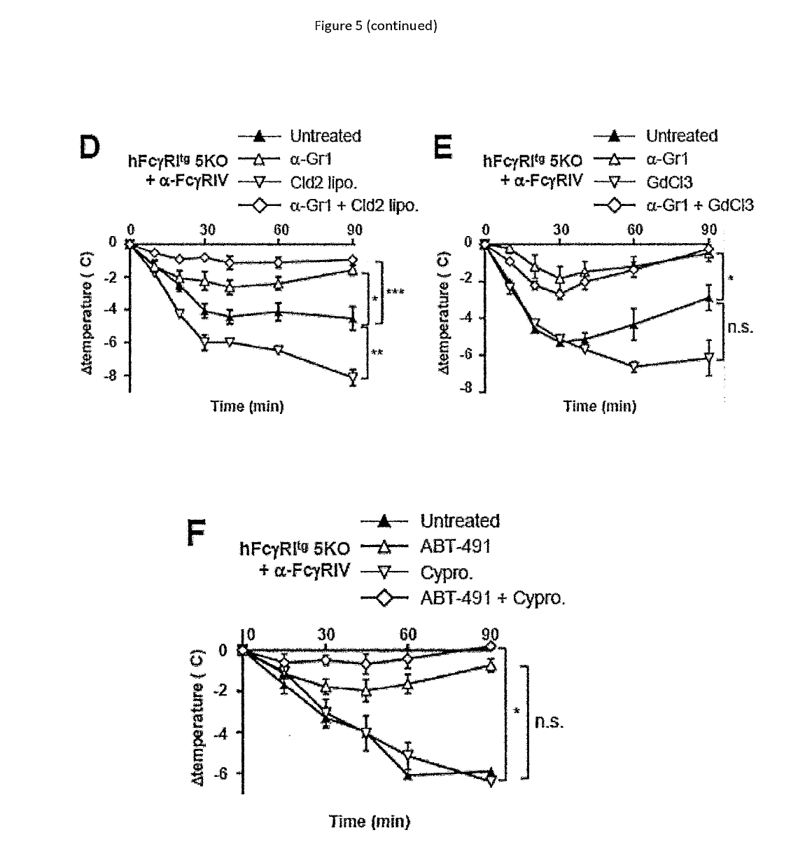

Because hFcγRI was sufficient to trigger PSA, it was then investigated if hFcγRI may also trigger active systemic anaphylaxis (ASA). ASA was induced by an i.v. antigen (BSA) challenge in mice repeatedly immunized with the same antigen in Freund's adjuvant (first immunization in complete, second and third immunization in incomplete Freund's adjuvant). This protocol induced a strong body temperature decrease in hFcγRI^{tg} 5KO mice, but not in 5KO mice, when pre-treated with anti-FcγRIV mAbs (FIG. 5A); what was termed “hFcγRI-dependent ASA”. Supporting this result, treatment with anti-hFcγRI.1 blocking mAb inhibited ASA-induced temperature drop (FIG. 5B) and mortality in hFcγR^{tg} 5KO mice. Blocking both hFcγRI and FcγRIV further inhibited ASA-induced temperature drop in these mice (FIG. 5B). hFcγRI is therefore sufficient to trigger active systemic anaphylaxis in transgenic mice.

Both effector cell types that express hFcγRI (i.e., monocytes/macrophages and neutrophils) can potentially contribute to ASA. hFcγRI-dependent ASA was strongly inhibited by neutrophil depletion following injection of anti-Gr1 mAbs (FIG. 5C).

Because this rat IgG2b anti-Gr1 mAb injection may lead to activation and depletion of complement components due to in vivo immune complex formation, it was investigated if the inhibition of hFcγRI-mediated active anaphylaxis following anti-Gr1 mAb treatment relied on complement. A dose of cobra venom factor (CVF) that inactivates both C3 and C5 components of the complement did neither prevent hFcγRI-mediated active anaphylaxis nor its inhibition following anti-Gr1 mAb injections (not shown). Therefore, the inhibition of anaphylaxis following anti-Gr1 mAb injection is dependent on neutrophil depletion per se, and not on complement. Surprisingly, neither monocyte/macrophage depletion following toxic liposomes injection (FIG. 5D), nor inhibition of monocyte/macrophage function following gadolinium injection (FIG. 5E) reduced hFcγRI-dependent ASA. Unexpectedly, the injection of toxic liposomes or of gadolinium rather increased hFcγRI-induced hypothermia. The depletion or inhibition of monocytes/macrophages, when combined with the depletion of neutrophils had, however, a tendency to increase the protection from hFcγRI-dependent ASA (FIG. 5D-E). Neutrophils and, possibly to a minor extent, monocytes/macrophages therefore contribute to hFcγRI-dependent ASA. Mediators released and/or secreted by these activated cell types should therefore be responsible for the anaphylactic shock observed. Among them, PAF was shown to be responsible for neutrophil-dependent ASA and for macrophage-dependent ASA, whereas histamine was shown to be responsible for mast cell-dependent anaphylaxis. The PAF-R antagonist ABT-491, but not the histamine and serotonin receptor antagonist cyproheptadine, markedly reduced hFcγRI-dependent temperature drop (FIG. 5F) and mortality (not shown). PAF therefore accounts for hFcγRI-dependent ASA. The conjunction of both antagonists, however, further reduced hFcγRI-dependent ASA (FIG. 5F). Noticeably, in addition to mast cells and basophils, neutrophils have been reported to be able to release histamine but not serotonin, suggesting that histamine released by neutrophils might, to a minor extent, contribute to hFcγRI-dependent ASA.

Monocytes/Macrophages Mediate hFcγRI-Dependent Thrombocytopenia

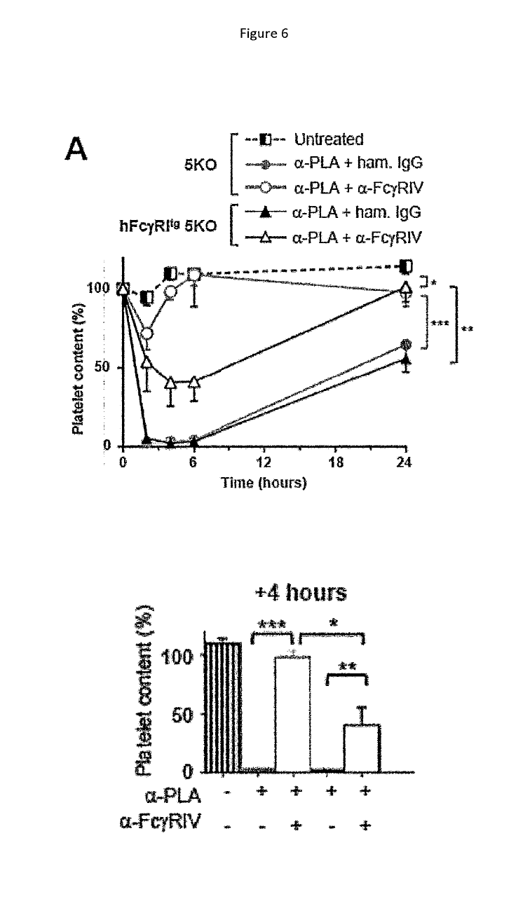

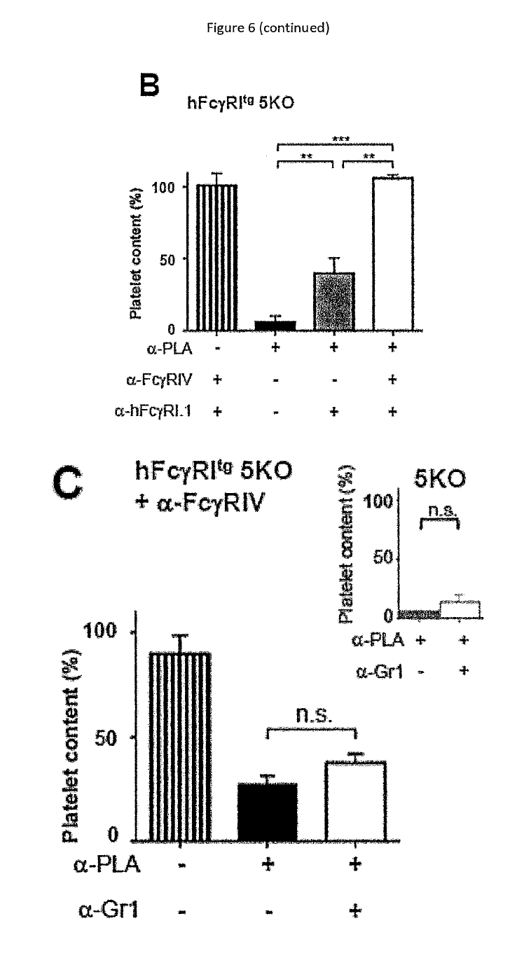

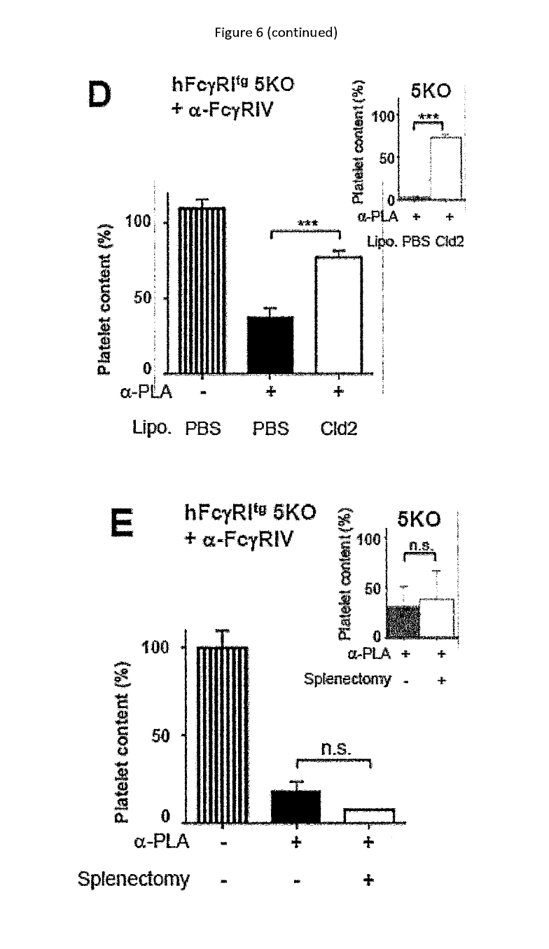

It was next investigated if, in addition to exerting pro-inflammatory and pro-anaphylactic properties, hFcγRI may also exert phagocytic properties in vivo using a murine model of thrombocytopenia. Immune Thrombocytopenic Purpura (ITP) can be induced by injecting intravenously anti-platelet antibodies (reminiscent of autoantibodies found in ITP patients) and by following circulating platelet consumption. ITP could be induced following injection of mouse IgG2a anti-platelet mouse IgG2a mAb both in hFcγRI^{tg} 5KO mice and in 5KO mice. FcγRIV blockade prevented ITP in 5KO mice, but reduced platelet consumption less than 50% in hFcγRI^{tg} 5KO mice (FIG. 6A,B). The remaining platelet consumption was hFcγRI-dependent, as it was prevented by a further hFcγRI blockade (FIG. 6B). hFcγRI-dependent ITP was not affected by neutrophil depletion (FIG. 6C), but was significantly inhibited by monocyte/macrophage depletion (FIG. 6D). Noticeably, splenectomy had no significant effect on hFcγRI-dependent ITP (FIG. 6E), suggesting that other hFcγRI-expressing macrophages than splenic macrophages contribute to platelet clearance in this model. Liver macrophages, i.e. Kupffer cells, which belong to the mononuclear phagocyte system express hFcγRI in hFcγRI^{tg} 5KO mice (FIG. 1A), could be responsible for platelet consumption in this model.

### Discussion

This work suggests that although hFcγRI is characterized as a high-affinity receptor for IgG, hFcγRI is readily available in vivo to bind IgG-immune complexes or IgG-opsonized targets. Despite its potential saturation by IgG in vivo, hFcγRI is indeed sufficient to mediate proinflammatory and pro-anaphylactic, leading to autoimmune and allergic reactions, respectively, in transgenic mice. Whereas both neutrophils and monocytes/macrophages are responsible for hFcγRI-induced autoimmune arthritis, neutrophils contributed predominantly to hFcγRI-induced anaphylaxis, and monocytes/macrophages contributed predominantly to hFcγRI-induced autoimmune thrombocytopenia.

This report supports the notion that being of high or of low affinity for IgG, FcγRs engaged by a given multivalent ligand and expressed by a given cell will induce with comparable kinetics the activation of that cell and consequently in vivo responses. It follows that the ability of high-affinity FcγRs to bind monomeric IgG has no detectable consequence in vivo. One could therefore consider that high-affinity FcγRs remain as unoccupied as low-affinity FcγRs in vivo. Nevertheless, the high concentration of circulating IgG favors the hypothesis that at any given time a proportion of high-affinity, but also of low-affinity, FcγRs are interacting with IgG. Low-affinity and high-affinity FcγRs were indeed reported to bind monomeric IgG with a half-life of the interaction varying from less than 1 minute to more than 10 minutes, respectively. The half-life of the interaction between hFcγRI and hIgG1 was reported to be 14 minutes in vitro. Results obtained in vivo nevertheless suggest that these half-lives are sufficiently short to allow low- and high-affinity FcγRs to bind IgG-immune complexes and to induce cell activation.

It was surprisingly found that hFcγRI can induce several allergy-related reactions in hFcγRI^{tg} mice. In the model of airway inflammation, hFcγRI triggered neutrophil infiltration, hemorrhage and MPO production in the alveolar space, symptoms that are reminiscent with those found in patients. hFcγRI was also able to induce passive systemic anaphylaxis when triggered by divalent or multivalent ligands, as well as ASA. Similarly as ASA in wt mice, hFcγRI-induced ASA relied predominantly on neutrophils and PAF. hFcγRI may be a key player in allergic and anaphylactic reactions in humans when allergen-specific IgG are present.

hFcγRI has been reported to allow antigen targeting to dendritic cells to enhance antigen presentation and it has been shown here that hFcγRI contributes to the induction of several inflammatory models in hFcγRI^{tg} mice. The mouse homolog of FcγRI, mFcγRI, is also expressed on dendritic cells and has been reported to play similar roles than hFcγRI in enhancing antigen presentation of IgG-bound antigen (Jonsson F. et al, J. Clin. Invest. 2011, 121(4):1484-1496). However, mFcγRI was not detected on monocyte or macrophage subsets nor on neutrophils. The absence of mFcγRI on effector cells suggest that its main activity may be to favor antigen presentation by and activation of dendritic cells, in agreement with its contributions reported following active immunization protocols. mFcγRI may therefore be a functional homolog of hFcγRI when considering dendritic cells only. When considering monocytes, macrophages and neutrophils, however mFcγRIV that does not exist in humans may be a functional homolog of hFcγRI. Like hFcγRI (this report), mFcγRIV is indeed expressed on these cell subsets and was reported to contribute to anaphylaxis (Jonsson F. et al, J. Clin. Invest. 2011; 121(4):1484-1496), arthritis (Mancardi et al, J. Immunol 2011; 186(4):1899-1903), airway inflammation (Skokowa J, et al. J Immunol. 2005, 174(5):3041-3050) and thrombocytopenia (Jonsson F. et al, J. Clin. Invest. 2011; 121(4):1484-1496). hFcγRI therefore recapitulates in humans the roles played in mice by mFcγRI on dendritic cells to favor antigen presentation and cell activation, and by mFcγRIV on monocytes/macrophages and neutrophils to trigger effector (pro-inflammatory) reactions.

It is reported here that hFcγRI can induce several mouse models of auto-immune and allergic reactions, and can therefore be considered as a potential pro-inflammatory and pro-anaphylactic activating IgG receptor in humans.

An anti-hFcγRI blocking mAb prevented hFcγRI-dependent models of autoimmunity and allergy, and may thus be useful in human pathologies.

Finally these results indicate that hFcγRI, and potentially other high-affinity FcRs, are either not occupied/saturated by IgG in vivo or if they are, this comes without functional consequence on their ability to mediate anti-tumor activities and pro-inflammatory and pro-anaphylactic properties.

### II. Role of Anti-hFcγRIIA mAb IV.3. In the Treatment of Inflammatory-Related Diseases

Efficient Blockade of the Human FcγRIIA Receptor

The anti-FcγRIIA monoclonal antibody IV.3 is easily obtainable by hybridoma sold by ATCC. It has V_{H} and V_{L} sequences as shown in SEQ ID NOs: 17 and 18 respectively. The blocking ability of IV.3 mAb towards human FcγRIIA has been reported (Looney R J, et al. J Immunol. 1986; 136(5):1641-1647).

II.1. Role of Anti-hFcγRIIA mAb IV.3. in the Treatment of Anaphylaxis

These results have been described in Jonsson et al, Blood 2012 (119:2533-2544).

Briefly, active systemic anaphylaxis (ASA) was induced by an IV antigen challenge in mice immunized with the same antigen. To analyze the capacity of FcγRIIA to induce ASA, a transgenic mouse models was developed, expressing human FcγRIIA under the control of its own promoter, and deficient for endogenous FcRs. FcγRII^{tg} mice express FcγRIIA not only on neutrophils, but also on eosinophils, monocytes, macrophages, and weakly on basophils. FcγRII^{tg} mice therefore reproduce the expression pattern found in humans. Surprisingly, IV injections of anti-FcγRIIA blocking mAbs abolished ASA-induced temperature drop and mortality in FcRγIIA mice immunized in Freund's adjuvant or in Alum.

To investigate the potential of FcγRIIA to induce passive systemic anaphylaxis (PSA), multivalent (IgG-immune complexes) ligands were used. We crossed FcγRII^{tg} mice to FcγRI/FcγRIIB/FcγRIIIA^{-/-} (3KO) mice or to FcγRI/FcγRIIB/FcγRIIIA^{-/-} FcεRI/FcεIIII^{-/-} (5KO) mice. 3KO and 5KO mice lack all IgG receptors except the activating IgG2 receptor FcγRIV whereas FcRγ^{-/-} mice lack all IgG receptors except the inhibitory IgG1/IgG2 receptor FcγRIIB. An i.v. injection of monoclonal IgG1- or polyclonal IgG-immune complexes induced a significant temperature drop in 3KOIIA mice, but not in 3KO mice. Pretreatment with anti-FcγRIIA mAb IV.3 abolished these temperature drops in 3KOIIA mice.

II.2. Role of Anti-hFcγRIIA mAb IV.3. in the Treatment of Passive Airway Inflammation

FcγRIIA is expressed in human lung tissue, but also in lung sections, and on alveolar macrophages from 3KOIIA mice.

We used a model of airway inflammation that consists of an IV injection of OVA and of an intranasal injection of anti-OVA rabbit serum, presumably forming ICs in vivo. Preformed OVA-anti-OVA rabbit serum ICs could bind to CHO cells expressing FcγRIIA, but not to untransfected CHO cells. CD11c⁺/Gr1⁻ alveolar macrophages represent more than 90% of the cells present in the alveolar space, as detected in broncho-alveolar lavages (BAL) of FcRγ^{-/-}, FcRγ^{-/-}IIA, and WT mice. Concomitant intranasal instillation of anti-OVA rabbit serum and intravenous injection of OVA induced a massive infiltration of CD11c⁺/Gr1⁻ cells (>80% of BAL content) in WT and in FcRγ^{-/-}IIA mice, but not in FcRγ^{-/-} mice (5% CD11c⁺/Gr1⁻ granulocytes). FcγRIIA therefore induces granulocytes recruitment to the lung, and can replace endogenous FcRγ-associated activating FcRs. Total cell numbers in the BAL were unchanged at t=3 hours after challenge, but increased starting t=6 hours and reached 5 times the background value at t=16 hours in FcRγ^{-/-}IIA mice, but not in FcRγ^{-/-} mice. Granulocyte numbers in BAL represented most of this increase, whereas alveolar macrophage numbers did not vary statistically along the time course.

Myeloperoxidase, which is mainly produced by neutrophils and by inflammatory macrophages in vivo, was detected at t=16 hours postchallenge in FcRγ^{-/-}IIA mice, but not in FcRγ^{-/-} mice. Similar results were obtained when analyzing the hemorrhage score that reflects lung tissue damage. KC, a chemokine produced by macrophages that can attract neutrophils to the site of inflammation, was found in BAL fluid of FcRγ^{-/-}IIA and to a lesser extent in FcRγ^{-/-} mice, as early as 3 hours after inoculation of antibody and antigen. This result suggests that alveolar macrophages are activated after FcγRIIA aggregation by IgG-immune complexes, and release KC before neutrophil accumulation in the broncho-alveolar space, in agreement with the dependency on alveolar macrophages reported for this disease model. Supporting this hypothesis, purified alveolar macrophages from FcRγ^{-/-}IIA mice, but not from FcRγ^{-/-} mice, secreted KC ex vivo after IgG-IC or anti-FcγRIIA mAb stimulation. Similar results were obtained when analyzing MIP-1α secretion, suggesting that FcγRIIA-triggered alveolar macrophages contribute to chemokine-induced granulocyte recruitment to the lung. FcγRIIA can therefore induce airway inflammation characterized by granulocyte infiltration in a passive antibody-dependent mouse model.

II.3. Role of Anti-hFcγRIIA mAb IV.3. in the Treatment of Arthritis

The mice disclosed in Jonsson et al, Blood 2012 were used. They carry the human FcγRIIA receptor but no endogenous IgG receptors (FcRγ^{-/-} background (γ^{-/-} IIA)).

The K/B×N Arthritis model defined in part I. has been used. hFcγRIIA^{tg} FcRγ^{-/-} mice, but not non-transgenic FcRγ^{-/-} littermates, developed arthritis following K/B×N serum injection (FIG. 9A). Anti-TNF-α blocking mAbs had no effect on hFcγRIIA-dependent K/B×N arthritis (FIG. 9B), in agreement with reports using wt mice. Blocking anti-FcγRIIA IV.3 mAbs, but not control IgG (isotype control of mAb IV.3) abolished arthritis in FcγRII^{tg} FcRγ^{-/-} mice (FIG. 9C). In comparison, a clinical dose (1 g/kg) of human intravenous immunoglobulins (IVIG), known to possess anti-inflammatory activities in this model (Bruhns P et al, Immunity 2003; 18(4):573-581) reduced, but did not abolish, arthritic symptoms in FcγRIIA^{tg} FcRγ^{-/-} mice.

Altogether, these results demonstrate that hFcγRIIA is sufficient to induce K/B×N passive arthritis that can be abolished by anti-FcγRIIA mAb IV.3 treatment, and significantly inhibited following IVIG treatment.

II.4. Role of Anti-FcγRIIA mAb IV.3 in the Treatment of Thrombocytopenia

We investigated if the property of hFcγRIIA to induce thrombocytopenia in the presence of anti-platelet IgG antibodies (reported by McKenzie S E, J Immunol. 1999; 162(7):4311-4318) may be inhibited by anti-hFcγRIIA mAb IV.3. We used a mouse model of immune thrombocytopenic Purpura (ITP) that can be induced by injecting intravenously anti-platelet antibodies (reminiscent of autoantibodies found in ITP patients) and by following circulating platelet consumption. ITP could be induced following injection of mouse Ig^{G2}a anτ^{l-p}platelet 6A6 mAb in hFcγRII^{tg} FcRγ^{-/-} mice, leading to >75% reduction in circulating plateλ^{et} numbers (FIG. 8), but not in FcRγ′ mice (data not shown). hFcγRIIA blockade induced by i.v. injection of mAb IV.3 reduced platelet consumption to ≈60% (FIG. 8).

hFcγRIIA-dependent ITP can thus be inhibited by treatment with IV.3 mAb.

Higher doses of IV.3 mAb may allow recovering of normal platelet numbers in this model.

1-15. (canceled)

16. An antibody or a functional fragment thereof, which binds and blocks the human FcγRI receptor, said antibody comprising six Complementary Determining Regions (CDRs) consisting of SEQ ID NO:1-6.

17. The antibody of claim 16, comprising:
a) a heavy chain comprising three CDRs having the following amino acid sequences:
(SEQ ID NO: 1)
i) the heavy chain CDR1: GFSLTTYG;
(SEQ ID NO: 2)
ii) the heavy chain CDR2: IWSGGST;
(SEQ ID NO: 3)
iii) the heavy chain CDR3: AREWFAY,
and
b) a light chain comprising three CDRs having the following amino acid sequences:
(SEQ ID NO: 4)
i) the light chain CDR1: ENIYSY;
(SEQ ID NO: 5)
ii) the light chain CDR2: SAK;
(SEQ ID NO: 6)
iii) the light chain CDR3: QHHYGTPYT.

18. The antibody of claim 16, comprising a heavy chain variable region (V_{H}) having the amino acid sequence SEQ ID NO: 9.

19. The antibody of claim 16, comprising a light chain variable region (V_{L}) having the amino acid sequence SEQ ID NO: 10.

20. A humanized antibody or a functional fragment thereof, comprising the CDRs as defined in any one of claim 16.

21. The humanized antibody of claim 20, or fragment thereof, comprising the sequences SEQ ID NO:19 and/or SEQ ID NO:20.

22. A method for preventing and/or treating IgG antibody-dependent inflammatory and autoimmune disorders, said method comprising administering the antibody of claim 16 to a subject.

23. The method of claim 22, wherein said disorder is chosen in the group consisting of: arthritic symptoms, allergic reactions, lupus and antibody-nephritis.

24. The method of claim 22, wherein said inflammatory disorder is rheumatoid arthritis.

25. The method of claim 22, wherein said inflammatory disorder is anaphylaxis.

26. A method for preventing and/or treating thrombocytopenia said method comprising administering the antibody of claim 16 to a subject.

27. A therapeutic substance combination product containing the antibody or fragment thereof as defined in claim 16, and a compound blocking the human FcγRIIA receptor.

28. The therapeutic substance combination product of claim 27, wherein said FcγRIIA blocking-compound is a monoclonal antibody or a functional fragment thereof.

29. The therapeutic substance combination product of claim 27, wherein said FcγRIIA blocking-compound is a monoclonal antibody comprising:
a) a heavy chain comprising three CDRs having the following amino acid sequences:
(SEQ ID NO: 11)
i) the heavy chain CDR1: GYTFTNYG;
(SEQ ID NO: 12)
ii) the heavy chain CDR2: LNTYTGES;
(SEQ ID NO: 13)
iii) the heavy chain CDR3: ARGDYGYDDPLDY,
and
b) a light chain comprising three CDRs having the following amino acid sequences:
(SEQ ID NO: 14)
i) the light chain CDR1: KSLLHTNGNTY;
(SEQ ID NO: 15)
ii) the light chain CDR2: RMSV;
(SEQ ID NO: 16)
iii) the light chain CDR3: MQHLEYPLT.

30. The therapeutic substance combination product of claim 27, wherein said FcγRIIA blocking-compound is a monoclonal antibody comprising a heavy chain variable region (V_{H}) having the amino acid sequence SEQ ID NO: 17 and/or a light chain variable region (V_{L}) having the amino acid sequence SEQ ID NO: 18.

31. A method for preventing and/or treating IgG antibody-dependent inflammatory and autoimmune disorders, said method comprising simultaneous, separate or sequential administering of the therapeutic substance combination product as defined in claim 27 to a subject.

32. The method of claim 31, wherein said inflammatory disorder is chosen among: arthritic symptoms, allergic reactions, lupus and antibody-nephritis.

33. The method of claim 31, wherein said inflammatory disorder is rheumatoid arthritis or anaphylaxis.

34. A method for preventing and/or treating thrombocytopenia, said method comprising simultaneous, separate or sequential administering of the therapeutic substance combination product as defined in claim 27 to a subject.
